SEQUENCE LISTING (28 sequences)

SEQ ID NO: 1 - protein, 735 aa, Homo sapiens
ALQQILGTKKKYFSTCKNWYKKSICGQKTTVLYECCPGYMRMEGMKGCPAVLPIDHVYGTLGIVGATTTQRYSDASKLREEIEGKGSFTYFAPSNEAWDNLDSDIRRGLESNVNVELLNALHSHMINKRMLTKDLKNGMIIPSMYNNLGLFINHYPNGVVTVNCARIIHGNQIATNGVVHVIDRVLTQIGTSIQDFIEAEDDLSSFRAAAITSDILEALGRDGHFTLFAPTNEAFEKLPRGVLERIMGDKVASEALMKYHILNTLQCSESIMGGAVFETLEGNTIEIGCDGDSITVNGIKMVNKKDIVTNNGVIHLIDQVLIPDSAKQVIELAGKQQTTFTDLVAQLGLASALRPDGEYTLLAPVNNAFSDDTLSMDQRLLKLILQNHILKVKVGLNELYNGQILETIGGKQLRVFVYRTAVCIENSCMEKGSKQGRNGAIHIFREIIKPAEKSLHEKLKQDKRFSTFLSLLEAADLKELLTQPGDWTLFVPTNDAFKGMTSEEKEILIRDKNALQNIILYHLTPGVFIGKGFEPGVTNILKTTQGSKIFLKEVNDTLLVNELKSKESDIMTTNGVIHVVDKLLYPADTPVGNDQLLEILNKLIKYIQIKFVRGSTFKEIPVTVYKPIIKKYTKIIDGVPVEITEKETREERIITGPEIKYTRISTGGGETEETLKKLLQEEVTKVTKFIEGGDGHLFEDEEIKRLLQGDTPVRKLQANKKVQGSRRRLREGRSQ

SEQ ID NO: 2 - DNA, 3130 bp, Homo sapiens
gccagttctcttcggggactaactgcaacggagagactcaagatgattccctttttacccatgttttctctactattgctgcttattgttaaccctataaacgccaacaatcattatgacaagatcttggctcatagtcgtatcaggggtcgggaccaaggcccaaatgtctgtgcccttcaacagattttgggcaccaaaaagaaatacttcagcacttgtaagaactggtataaaaagtccatctgtggacagaaaacgactgtgttatatgaatgttgccctggttatatgagaatggaaggaatgaaaggctgcccagcagttttgcccattgaccatgtttatggcactctgggcatcgtgggagccaccacaacgcagcgctattctgacgcctcaaaactgagggaggagatcgagggaaagggatccttcacttactttgcaccgagtaatgaggcttgggacaacttggattctgatatccgtagaggtttggagagcaacgtgaatgttgaattactgaatgctttacatagtcacatgattaataagagaatgttgaccaaggacttaaaaaatggcatgattattccttcaatgtataacaatttggggcttttcattaaccattatcctaatggggttgtcactgttaattgtgctcgaatcatccatgggaaccagattgcaacaaatggtgttgtccatgtcattgaccgtgtgcttacacaaattggtacctcaattcaagacttcattgaagcagaagatgacctttcatcttttagagcagctgccatcacatcggacatattggaggcccttggaagagacggtcacttcacactctttgctcccaccaatgaggcttttgagaaacttccacgaggtgtcctagaaaggatcatgggagacaaagtggcttccgaagctcttatgaagtaccacatcttaaatactctccagtgttctgagtctattatgggaggagcagtctttgagacgctggaaggaaatacaattgagataggatgtgacggtgacagtataacagtaaatggaatcaaaatggtgaacaaaaaggatattgtgacaaataatggtgtgatccatttgattgatcaggtcctaattcctgattctgccaaacaagttattgagctggctggaaaacagcaaaccaccttcacggatcttgtggcccaattaggcttggcatctgctctgaggccagatggagaatacactttgctggcacctgtgaataatgcattttctgatgatactctcagcatggatcagcgcctccttaaattaattctgcagaatcacatattgaaagtaaaagttggccttaatgagctttacaacgggcaaatactggaaaccatcggaggcaaacagctcagagtcttcgtatatcgtacagctgtctgcattgaaaattcatgcatggagaaagggagtaagcaagggagaaacggtgcgattcacatattccgcgagatcatcaagccagcagagaaatccctccatgaaaagttaaaacaagataagcgctttagcaccttcctcagcctacttgaagctgcagacttgaaagagctcctgacacaacctggagactggacattatttgtgccaaccaatgatgcttttaagggaatgactagtgaagaaaaagaaattctgatacgggacaaaaatgctcttcaaaacatcattctttatcacctgacaccaggagttttcattggaaaaggatttgaacctggtgttactaacattttaaagaccacacaaggaagcaaaatctttctgaaagaagtaaatgatacacttctggtgaatgaattgaaatcaaaagaatctgacatcatgacaacaaatggtgtaattcatgttgtagataaactcctctatccagcagacacacctgttggaaatgatcaactgctggaaatacttaataaattaatcaaatacatccaaattaagtttgttcgtggtagcaccttcaaagaaatccccgtgactgtctataagccaattattaaaaaatacaccaaaatcattgatggagtgcctgtggaaataactgaaaaagagacacgagaagaacgaatcattacaggtcctgaaataaaatacactaggatttctactggaggtggagaaacagaagaaactctgaagaaattgttacaagaagaggtcaccaaggtcaccaaattcattgaaggtggtgatggtcatttatttgaagatgaagaaattaaaagactgcttcagggagacacacccgtgaggaagttgcaagccaacaaaaaagttcaaggatctagaagacgattaagggaaggtcgttctcagtgaaaatccaaaaaccagaaaaaaatgtttatacaaccctaagtcaataacctgaccttagaaaattgtgagagccaagttgacttcaggaactgaaacatcagcacaaagaagcaatcatcaaataattctgaacacaaatttaatatttttttttctgaatgagaaacatgagggaaattgtggagttagcctcctgtggtaaaggaattgaagaaaatataacaccttacaccctttttcatcttgacattaaaagttctggctaactttggaatccattagagaaaaatccttgtcaccagattcattacaattcaaatcgaagagttgtgaactgttatcccattgaaaagaccgagccttgtatgtatgttatggatacataaaatgcacgcaagccattatctctccatgggaagctaagttataaaaataggtgcttggtgtacaaaactttttatatcaaaaggctttgcacatttctatatgagtgggtttactggtaaattatgttattttttacaactaattttgtactctcagaatgtttgtcatatgcttcttgcaatgcatattttttaatctcaaacgtttcaataaaaccatttttcagatataaagagaattacttcaaattgagtaattcagaaaaactcaagatttaagttaaaaagtggtttggacttgggaacaggactttatacctcttttactgtaacaagtactcattaaaggaaattgaatgaaa

SEQ ID NO: 3 - protein, 807 aa, Homo sapiens
MGTSHPAFLVLGCLLTGLSLILCQLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKATSELDLEMEALKTVYKSGETIVVTCAVFNNEVVDLQWTYPGEVKGKGITMLEEIKVPSIKLVYTLTVPEATVKDSGDYECAARQATREVKEMKKVTISVHEKGFIEIKPTFSQLEAVNLHEVKHFVVEVRAYPPPRISWLKNNLTLIENLTEITTDVEKIQEIRYRSKLKLIRAKEEDSGHYTIVAQNEDAVKSYTFELLTQVPSSILDLVDDHHGSTGGQTVRCTAEGTPLPDIEWMICKDIKKCNNETSWTILANNVSNIITEIHSRDRSTVEGRVTFAKVEETIAVRCLAKNLLGAENRELKLVAPTLRSELTVAAAVLVLLVIVIISLIVLVVIWKQKPRYEIRWRVIESISPDGHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLSHHPEKPKKELDIFGLNPADESTRSYVILSFENNGDYMDMKQADTTQYVPMLERKEVSKYSDIQRSLYDRPASYKKKSMLGWAGGVGTRCQRNLSHAQAQALLAGRPLLSGLP

SEQ ID NO: 4 - DNA, 2856 bp, Homo sapiens
attactgttggagctacagggagagaaacagaggaggagactgcaagagatcattggaggccgtgggcacgctctttactccatgtgtgggacattcattgcggaataacatcggaggagaagtttcccagagctatggggacttcccatccggcgttcctggtcttaggctgtcttctcacagggctgagcctaatcctctgccagctttcattaccctctatccttccaaatgaaaatgaaaaggttgtgcagctgaattcatccttttctctgagatgctttggggagagtgaagtgagctggcagtaccccatgtctgaagaagagagctccgatgtggaaatcagaaatgaagaaaacaacagcggcctttttgtgacggtcttggaagtgagcagtgcctcggcggcccacacagggttgtacacttgctattacaaccacactcagacagaagagaatgagcttgaaggcaggcacatttacatctatgtgccagacccagatgtagcctttgtacctctaggaatgacggattatttagtcatcgtggaggatgatgattctgccattataccttgtcgcacaactgatcccgagactcctgtaaccttacacaacagtgagggggtggtacctgcctcctacgacagcagacagggctttaatgggaccttcactgtagggccctatatctgtgaggccaccgtcaaaggaaagaagttccagaccatcccatttaatgtttatgctttaaaagcaacatcagagctggatctagaaatggaagctcttaaaaccgtgtataagtcaggggaaacgattgtggtcacctgtgctgtttttaacaatgaggtggttgaccttcaatggacttaccctggagaagtgaaaggcaaaggcatcacaatgctggaagaaatcaaagtcccatccatcaaattggtgtacactttgacggtccccgaggccacggtgaaagacagtggagattacgaatgtgctgcccgccaggctaccagggaggtcaaagaaatgaagaaagtcactatttctgtccatgagaaaggtttcattgaaatcaaacccaccttcagccagttggaagctgtcaacctgcatgaagtcaaacattttgttgtagaggtgcgggcctacccacctcccaggatatcctggctgaaaaacaatctgactctgattgaaaatctcactgagatcaccactgatgtggaaaagattcaggaaataaggtatcgaagcaaattaaagctgatccgtgctaaggaagaagacagtggccattatactattgtagctcaaaatgaagatgctgtgaagagctatacttttgaactgttaactcaagttccttcatccattctggacttggtcgatgatcaccatggctcaactgggggacagacggtgaggtgcacagctgaaggcacgccgcttcctgatattgagtggatgatatgcaaagatattaagaaatgtaataatgaaacttcctggactattttggccaacaatgtctcaaacatcatcacggagatccactcccgagacaggagtaccgtggagggccgtgtgactttcgccaaagtggaggagaccatcgccgtgcgatgcctggctaagaatctccttggagctgagaaccgagagctgaagctggtggctcccaccctgcgttctgaactcacggtggctgctgcagtcctggtgctgttggtgattgtgatcatctcacttattgtcctggttgtcatttggaaacagaaaccgaggtatgaaattcgctggagggtcattgaatcaatcagcccagatggacatgaatatatttatgtggacccgatgcagctgccttatgactcaagatgggagtttccaagagatggactagtgcttggtcgggtcttggggtctggagcgtttgggaaggtggttgaaggaacagcctatggattaagccggtcccaacctgtcatgaaagttgcagtgaagatgctaaaacccacggccagatccagtgaaaaacaagctctcatgtctgaactgaagataatgactcacctggggccacatttgaacattgtaaacttgctgggagcctgcaccaagtcaggccccatttacatcatcacagagtattgcttctatggagatttggtcaactatttgcataagaatagggatagcttcctgagccaccacccagagaagccaaagaaagagctggatatctttggattgaaccctgctgatgaaagcacacggagctatgttattttatcttttgaaaacaatggtgactacatggacatgaagcaggctgatactacacagtatgtccccatgctagaaaggaaagaggtttctaaatattccgacatccagagatcactctatgatcgtccagcctcatataagaagaaatctatgttaggctgggctggtggagttggcacgagatgtcagaggaacctgagtcatgctcaggcccaagccctgttggcaggcagaccactgctttctggccttccgtgactatctgaaaaaaatcgtgaatggctagagctactcttcacttgctgaacattttcaaaaagaattgagaacttctggattaaattgccttcttcctcgaaaaccctgggacccttccagatgggactaactggggaaagtggacaagttacaaacaaagaaactcaaaggaaagtcattggcactgatctctaagatgctatcacatgtgattggtggttgattttattaacaaattataagcaaagtactacaaaggtggctttaaaaagaaaataaagcaattcacagaaacta

SEQ ID NO: 5 - protein, 757 aa, Homo sapiens
MLLRLLLAWAAAGPTLGQDPWAAEPRAACGPSSCYALFPRRRTFLEAWRACRELGGDLATPRTPEEAQRVDSLVGAGPASRLLWIGLQRQARQCQLQRPLRGFTWTTGDQDTAFTNWAQPASGGPCPAQRCVALEASGEHRWLEGSCTLAVDGYLCQFGFEGACPALQDEAGQAGPAVYTTPFHLVSTEFEWLPFGSVAAVQCQAGRGASLLCVKQPEGGVGWSRAGPLCLGTGCSPDNGGCEHECVEEVDGHVSCRCTEGFRLAADGRSCEDPCAQAPCEQQCEPGGPQGYSCHCRLGFRPAEDDPHRCVDTDECQIAGVCQQMCVNYVGGFECYCSEGHELEADGISCSPAGAMGAQASQDLGDELLDDGEDEEDEDEAWKAFNGGWTEMPGILWMEPTQPPDFALAYRPSFPEDREPQIPYPEPTWPPPLSAPRVPYHSSVLSVTRPVVVSATHPTLPSAHQPPVIPATHPALSRDHQIPVIAANYPDLPSAYQPGILSVSHSAQPPAHQPPMISTKYPELFPAHQSPMFPDTRVAGTQTTTHLPGIPPNHAPLVTTLGAQLPPQAPDALVLRTQATQLPIIPTAQPSLTTTSRSPVSPAHQISVPAATQPAALPTLLPSQSPTNQTSPISPTHPHSKAPQIPREDGPSPKLALWLPSPAPTAAPTALGEAGLAEHSQRDDRWLLVALLVPTCVFLVVLLALGIVYCTRCGPHAPNKRITDCYRWVIHAGSKSPTEPMPPRGSLTGVQTCRTSV

SEQ ID NO: 6 - DNA, 2551 bp, Homo sapiens
agtccgggggcatcgcgatgctgctgcgcctgttgctggcctgggcggccgcagggcccacactgggccaggacccctgggctgctgagccccgtgccgcctgcggccccagcagctgctacgctctcttcccacggcgccgcaccttcctggaggcctggcgggcctgccgcgagctggggggcgacctggccactcctcggacccccgaggaggcccagcgtgtggacagcctggtgggtgcgggcccagccagccggctgctgtggatcgggctgcagcggcaggcccggcaatgccagctgcagcgcccactgcgcggcttcacgtggaccacaggggaccaggacacggctttcaccaactgggcccagccagcctctggaggcccctgcccggcccagcgctgtgtggccctggaggcaagtggcgagcaccgctggctggagggctcgtgcacgctggctgtcgacggctacctgtgccagtttggcttcgagggcgcctgcccggcgctgcaagatgaggcgggccaggccggcccagccgtgtataccacgcccttccacctggtctccacagagtttgagtggctgcccttcggctctgtggccgctgtgcagtgccaggctggcaggggagcctctctgctctgcgtgaagcagcctgagggaggtgtgggctggtcacgggctgggcccctgtgcctggggactggctgcagccctgacaacgggggctgcgaacacgaatgtgtggaggaggtggatggtcacgtgtcctgccgctgcactgagggcttccggctggcagcagacgggcgcagttgcgaggacccctgtgcccaggctccgtgcgagcagcagtgtgagcccggtgggccacaaggctacagctgccactgtcgcctgggtttccggccagcggaggatgatccgcaccgctgtgtggacacagatgagtgccagattgccggtgtgtgccagcagatgtgtgtcaactacgttggtggcttcgagtgttattgtagcgagggacatgagctggaggctgatggcatcagctgcagccctgcaggggccatgggtgcccaggcttcccaggacctcggagatgagttgctggatgacggggaggatgaggaagatgaagacgaggcctggaaggccttcaacggtggctggacggagatgcctgggatcctgtggatggagcctacgcagccgcctgactttgccctggcctatagaccgagcttcccagaggacagagagccacagataccctacccggagcccacctggccacccccgctcagtgcccccagggtcccctaccactcctcagtgctctccgtcacccggcctgtggtggtctctgccacgcatcccacactgccttctgcccaccagcctcctgtgatccctgccacacacccagctttgtcccgtgaccaccagatccccgtgatcgcagccaactatccagatctgccttctgcctaccaacccggtattctctctgtctctcattcagcacagcctcctgcccaccagccccctatgatctcaaccaaatatccggagctcttccctgcccaccagtcccccatgtttccagacacccgggtcgctggcacccagaccaccactcatttgcctggaatcccacctaaccatgcccctctggtcaccaccctcggtgcccagctaccccctcaagccccagatgcccttgtcctcagaacccaggccacccagcttcccattatcccaactgcccagccctctctgaccaccacctccaggtcccctgtgtctcctgcccatcaaatctctgtgcctgctgccacccagcccgcagccctccccaccctcctgccctctcagagccccactaaccagacctcacccatcagccctacacatccccattccaaagccccccaaatcccaagggaagatggccccagtcccaagttggccctgtggctgccctcaccagctcccacagcagccccaacagccctgggggaggctggtcttgccgagcacagccagagggatgaccggtggctgctggtggcactcctggtgccaacgtgtgtctttttggtggtcctgcttgcactgggcatcgtgtactgcacccgctgtggcccccatgcacccaacaagcgcatcactgactgctatcgctgggtcatccatgctgggagcaagagcccaacagaacccatgccccccaggggcagcctcacaggggtgcagacctgcagaaccagcgtgtgatggggtgcagacccccctcatggagtatggggcgctggacacatggccggggctgcaccagggacccatgggggctgcccagctggacagatggcttcctgctccccaggcccagccagggtcctctctcaaccactagacttggctctcaggaactctgcttcctggcccagcgctcgtgaccaaggatacaccaaagcccttaagacctcagggggcgggtgctggggtcttctccaataaatggggtgtcaacctta

SEQ ID NO: 7 - protein, 235 aa, Homo sapiens
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 8 - DNA, 1101 bp, Homo sapiens
cctttcactttcggtctctggctgtcacccggcttggccccttccacacccaactggggcaagcctgacccggcgacaggaggcatgaggggcccccggccgaaatgacagtgctggcgccagcctggagcccaacaacctatctcctcctgctgctgctgctgagctcgggactcagtgggacccaggactgctccttccaacacagccccatctcctccgacttcgctgtcaaaatccgtgagctgtctgactacctgcttcaagattacccagtcaccgtggcctccaacctgcaggacgaggagctctgcgggggcctctggcggctggtcctggcacagcgctggatggagcggctcaagactgtcgctgggtccaagatgcaaggcttgctggagcgcgtgaacacggagatacactttgtcaccaaatgtgcctttcagcccccccccagctgtcttcgcttcgtccagaccaacatctcccgcctcctgcaggagacctccgagcagctggtggcgctgaagccctggatcactcgccagaacttctcccggtgcctggagctgcagtgtcagcccgactcctcaaccctgccacccccatggagtccccggcccctggaggccacagccccgacagccccgcagccccctctgctcctcctactgctgctgcccgtgggcctcctgctgctggccgctgcctggtgcctgcactggcagaggacgcggcggaggacaccccgccctggggagcaggtgccccccgtccccagtccccaggacctgctgcttgtggagcactgacctggccaaggcctcatcctgcggagccttaaacaacgcagtgagacagacatctatcatcccattttacaggggaggatactgaggcacacagaggggagtcaccagccagaggatgcatagcctggacacagaggaagttggctagaggccggtcccttccttgggcccctctcattccctccccagaatggaggcaacgccagaatccagcaccggccccatttacccaactctgtacaaagcccttgtccccatgaaattgtatataaatcatccttttctacca

SEQ ID NO: 9 - protein, 98 aa, Homo sapiens
MALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 10 - DNA, 683 bp, Homo sapiens
attcccagcctcacatcactcacaccttgcatttcacccctgcatcccagtcgccctgcagcctcacacagatcctgcacacacccagacagctggcgctcacacattcaccgttggcctgcctctgttcaccctccatggccctgctactggccctcagcctgctggttctctggacttccccagccccaactctgagtggcaccaatgatgctgaagactgctgcctgtctgtgacccagaaacccatccctgggtacatcgtgaggaacttccactaccttctcatcaaggatggctgcagggtgcctgctgtagtgttcaccacactgaggggccgccagctctgtgcacccccagaccagccctgggtagaacgcatcatccagagactgcagaggacctcagccaagatgaagcgccgcagcagttaacctatgaccgtgcagagggagcccggagtccgagtcaagcattgtgaattattacctaacctggggaaccgaggaccagaaggaaggaccaggcttccagctcctctgcaccagacctgaccagccaggacagggcctggggtgtgtgtgagtgtgagtgtgagcgagagggtgagtgtggtcagagtaaagctgctccacccccagattgcaatgctaccaataaagccgcctggtgtttacaactaa

SEQ ID NO: 11 - protein, 396 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 12 - DNA, 3545 bp, Homo sapiens
gccgccgccgccgtcgccgccgccggagtcctcgccccgccgcgctgcgcccggctcgcgctgcgctagtcgctccgcttcccacaccccgccggggactggcagccgccgccgcacatctgccgccacagcctccgccggctacccgaacgttctcggggccagcgccgagtggatcaccggggaccgcgaggcacccgcgcgccgcagaccccgcgcgggctggagcacccggcagagcgcgccacagcgccgtggcctctgctgcccgggctgcgccagagccgcggacgggcgcgcagagcgccggggactccggagccgatccctagcgccgcgatgcggagcacctactgcaggagatcgggggcctgggacgcgctggccgaggtgtgatcggaccccaggctagccacaaagggcacttggccccagggctaggagagcgaggggagagcacagccacccgcctcggcggcccgggactcggctcgactcgccggagaatgcgcccgaggacgacggggcgccagagccgcggtgctttcaactggcgagcgcgaatgggggtgcactggagtaaggcagagtgatgcgggggggcaactcgcctggcaccgagatcgccgccgtgcccttccctggacccggcgtcgcccaggatggctgccccgagccatgggccgcggcggagctagcgcggagcgcccgaccctcgacccccgagtcccggagccggccccgcgcggggccacgcgtccctcgggcgctggttcctaaggaggacgacagcaccagcttctcctttctcccttcccttccctgccccgcactcctccccctgctcgctgttgttgtgtgtcagcacttggctggggacttcttgaacttgcagggagaataacttgcgcaccccactttgcgccggtgcctttgccccagcggagcctgcttcgccatctccgagccccaccgcccctccactcctcggccttgcccgacactgagacgctgttcccagcgtgaaaagagagactgcgcggccggcacccgggagaaggaggaggcaaagaaaaggaacggacattcggtccttgcgccaggtcctttgaccagagtttttccatgtggacgctctttcaatggacgtgtccccgcgtgcttcttagacggactgcggtctcctaaaggtcgaccatggtggccgggacccgctgtcttctagcgttgctgcttccccaggtcctcctgggcggcgcggctggcctcgttccggagctgggccgcaggaagttcgcggcggcgtcgtcgggccgcccctcatcccagccctctgacgaggtcctgagcgagttcgagttgcggctgctcagcatgttcggcctgaaacagagacccacccccagcagggacgccgtggtgcccccctacatgctagacctgtatcgcaggcactcaggtcagccgggctcacccgccccagaccaccggttggagagggcagccagccgagccaacactgtgcgcagcttccaccatgaagaatctttggaagaactaccagaaacgagtgggaaaacaacccggagattcttctttaatttaagttctatccccacggaggagtttatcacctcagcagagcttcaggttttccgagaacagatgcaagatgctttaggaaacaatagcagtttccatcaccgaattaatatttatgaaatcataaaacctgcaacagccaactcgaaattccccgtgaccagacttttggacaccaggttggtgaatcagaatgcaagcaggtgggaaagttttgatgtcacccccgctgtgatgcggtggactgcacagggacacgccaaccatggattcgtggtggaagtggcccacttggaggagaaacaaggtgtctccaagagacatgttaggataagcaggtctttgcaccaagatgaacacagctggtcacagataaggccattgctagtaacttttggccatgatggaaaagggcatcctctccacaaaagagaaaaacgtcaagccaaacacaaacagcggaaacgccttaagtccagctgtaagagacaccctttgtacgtggacttcagtgacgtggggtggaatgactggattgtggctcccccggggtatcacgccttttactgccacggagaatgcccttttcctctggctgatcatctgaactccactaatcatgccattgttcagacgttggtcaactctgttaactctaagattcctaaggcatgctgtgtcccgacagaactcagtgctatctcgatgctgtaccttgacgagaatgaaaaggttgtattaaagaactatcaggacatggttgtggagggttgtgggtgtcgctagtacagcaaaattaaatacataaatatatatatatatatatattttagaaaaaagaaaaaaacaaacaaacaaaaaaaccccaccccagttgacactttaatatttcccaatgaagactttatttatggaatggaatggaaaaaaaaacagctattttgaaaatatatttatatctacgaaaagaagttgggaaaacaaatattttaatcagagaattattccttaaagatttaaaatgtatttagttgtacattttatatgggttcaaccccagcacatgaagtataatggtcagatttattttgtatttatttactattataaccactttttaggaaaaaaatagctaatttgtatttatatgtaatcaaaagaagtatcgggtttgtacataattttccaaaaattgtagttgttttcagttgtgtgtatttaagatgaaaagtctacatggaaggttactctggcaaagtgcttagcacgtttgcttttttgcagtgctactgttgagttcacaagttcaagtccagaaaaaaaaagtggataatccactctgctgactttcaagattattatattattcaattctcaggaatgttgcagagtgattgtccaatccatgagaatttacatccttattaggtggaatatttggataagaaccagacattgctgatctattatagaaactctcctcctgccccttaatttacagaaagaataaagcaggatccatagaaataattaggaaaacgatgaacctgcaggaaagtgaatgatggtttgttgttcttctttcctaaattagtgatcccttcaaaggggctgatctggccaaagtattcaataaaacgtaagatttcttcattattgatattgtggtcatatatatttaaaattgatatctcgtggccctcatcaagggttggaaatttatttgtgttttacctttacctcatctgagagctctttattctccaaagaacccagttttctaactttttgcccaacacgcagcaaaattatgcacatcgtgttttctgcccaccctctgttctctgacctatcagcttgcttttctttccaaggttgtgtgtttgaacacatttctccaaatgttaaacctatttcagataataaatatcaaatctctggca

SEQ ID NO: 13 - protein, 352 aa, Homo sapiens
MIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEVISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNST

SEQ ID NO: 14 - DNA, 1931 bp, Homo sapiens
ggaagatgcgagaaggcagaggaggagggagggagggaaggagcgcggagcccggcccggaagctaggtgagtgtggcatccgagctgagggacgcgagcctgagacgccgctgctgctccggctgagtatctagcttgtctccccgatgggattcccgtccaagctatctcgagcctgcagcgccacagtccccggccctcgcccaggttcactgcaaccgttcagaggtccccaggagctgctgctggcgagcccgctactgcagggacctatggagccattccgtagtgccatcccgagcaacgcactgctgcagcttccctgagcctttccagcaagtttgttcaagattggctgtcaagaatcatggactgttattatatgccttgttttctgtcaagacaccatgattcctggtaaccgaatgctgatggtcgttttattatgccaagtcctgctaggaggcgcgagccatgctagtttgatacctgagacggggaagaaaaaagtcgccgagattcagggccacgcgggaggacgccgctcagggcagagccatgagctcctgcgggacttcgaggcgacacttctgcagatgtttgggctgcgccgccgcccgcagcctagcaagagtgccgtcattccggactacatgcgggatctttaccggcttcagtctggggaggaggaggaagagcagatccacagcactggtcttgagtatcctgagcgcccggccagccgggccaacaccgtgaggagcttccaccacgaagaacatctggagaacatcccagggaccagtgaaaactctgcttttcgtttcctctttaacctcagcagcatccctgagaacgaggtgatctcctctgcagagcttcggctcttccgggagcaggtggaccagggccctgattgggaaaggggcttccaccgtataaacatttatgaggttatgaagcccccagcagaagtggtgcctgggcacctcatcacacgactactggacacgagactggtccaccacaatgtgacacggtgggaaacttttgatgtgagccctgcggtccttcgctggacccgggagaagcagccaaactatgggctagccattgaggtgactcacctccatcagactcggacccaccagggccagcatgtcaggattagccgatcgttacctcaagggagtgggaattgggcccagctccggcccctcctggtcacctttggccatgatggccggggccatgccttgacccgacgccggagggccaagcgtagccctaagcatcactcacagcgggccaggaagaagaataagaactgccggcgccactcgctctatgtggacttcagcgatgtgggctggaatgactggattgtggccccaccaggctaccaggccttctactgccatggggactgcccctttccactggctgaccacctcaactcaaccaaccatgccattgtgcagaccctggtcaattctgtcaattccagtatccccaaagcctgttgtgtgcccactgaactgagtgccatctccatgctgtacctggatgagtatgataaggtggtactgaaaaattatcaggagatggtagtagagggatgtgggtgccgctgagatcaggcagtccttgaggatagacagatatacacaccacacacacacaccacatacaccacacacacacgttcccatccactcacccacacactacacagactgcttccttatagctggacttttatttaaaaaaaaaaaaaaaaaaggaaaaaatccctaaacattcaccttgaccttatttatgactttacgtgcaaatgttttgaccatattgatcatatattttgacaaaatatatttataactacgtattaaaagaaaaaaataaaatgagtcattattttaaaggtaaa

SEQ ID NO: 15 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 16 - DNA, 2015 bp, Homo sapiens
cttttcgccaggggttgggactccgggtggcaggcgcccgggggaatcccagctgactcgctcactgccttcgaagtccggcgccccccgggagggaactgggtggccgcaccctcccggctgcggtggctgtcgccccccaccctgcagccaggactcgatggagaatccattccaatatatggccatgtggctctttggagcaatgttccatcatgttccatgctgctgacgtcacatggagcacagaaatcaatgttagcagatagccagcccatacaagatcgtattgtattgtaggaggcattgtggatggatggctgctggaaaccccttgccatagccagctcttcttcaatacttaaggatttaccgtggctttgagtaatgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttgattgcaattgattctttttaaagtgtttctgttattaacaaacatcactctgctgcttagacataacaaaacactcggcatttcaaatgtgctgtcaaaacaagtttttctgtcaagaagatgatcagaccttggatcagatgaactcttagaaatgaaggcagaaaaatgtcattgagtaatatagtgactatgaacttctctcagacttactttactcatttttttaatttattattgaaattgtacatatttgtggaataatgtaaaatgttgaataaaaatatgtacaagtgttgttttttaagttgcactgatattttacctcttattgcaaaatagcatttgtttaagggtgatagtcaaattatgtattggtggggctgggtaccaatgctgcaggtcaacagctatgctggtaggctcctgccagtgtggaaccactgactactggctctcattgacttccttactaagcatagcaaacagaggaagaatttgttatcagtaagaaaaagaagaactatatgtgaatcctcttctttatactgtaatttagttattgatgtataaagcaactgttatgaaataaagaaattgcaataactggcatataatgtccatcagtaaatcttggtggtggtggcaataataaacttctactgataggtagaatggtgtgcaagcttgtccaatcacggattgcaggccacatgcggcccaggacaactttgaatgtggcccaacacaaattcataaactttcatacatctcgtttttagctcatcagctatcattagcggtagtgtatttaaagtgtggcccaagacaattcttcttattccaatgtggcccagggaaatcaaaagattggatgcccctggtatagaaaactaatagtgacagtgttcatatttcatgctttcccaaatacaggtattttattttcacattctttttgccatgtttatataataataaagaaaaaccctgttgatttgttggagccattgttatctgacagaaaataattgtttatattttttgcactacactgtctaaaattagcaagctctcttctaatggaactgtaagaaagatgaaatatttttgttttattataaatttatttcaccttaa

SEQ ID NO: 17 - protein, 253 aa, Homo sapiens
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 18 - DNA, 1444 bp, Homo sapiens
atttcgctttcattttgggccgagctggaggcggcggggccgtcccggaacggctgcggccgggcaccccgggagttaatccgaaagcgccgcaagccccgcgggccggccgcaccgcacgtgtcaccgagaagctgatgtagagagagacacagaaggagacagaaagcaagagaccagagtcccgggaaagtcctgccgcgcctcgggacaattataaaaatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaaaaagcgaggtccctccaaaccgttgtcatttttataaaactttgaaatgaggaaactttgataggatgtggattaagaactagggagggggaaagaaggatgggactattacatccacatgatacctctgatcaagtatttttgacatttactgtggataaattgtttttaagttttcatgaatgaattgctaagaagggaaaatatccatcctgaaggtgtttttcattcactttaatagaagggcaaatatttataagctatttctgtaccaaagtgtttgtggaaacaaacatgtaagcataacttattttaaaatatttatttatataacttggtaatcatgaaagcatctgagctaacttatatttatttatgttatatttattaaattatttatcaagtgtatttgaaaaatatttttaagtgttctaaaaataaaagtattgaattaaagtga

SEQ ID NO: 19 - protein, 99 aa, Homo sapiens
MRLLAAALLLLLLALYTARVDGSKCKCSRKGPKIRYSDVKKLEMKPKYPHCEEKMVIITTKSVSRYRGQEHCLHPKLQSTKRFIKWYNAWNEKRRVYEE

SEQ ID NO: 20 - DNA, 1687 bp, Homo sapiens
gtggagagagcgcggagacaagcgcagagcgcagcgcacggccacagacagccctgggcatccaccgacggcgcagccggagccagcagagccggaaggcgcgccccgggcagagaaagccgagcagagctgggtggcgtctccgggccgccgctccgacgggccagcgccctccccatgtccctgctcccacgccgcgcccctccggtcagcatgaggctcctggcggccgcgctgctcctgctgctgctggcgctgtacaccgcgcgtgtggacgggtccaaatgcaagtgctcccggaagggacccaagatccgctacagcgacgtgaagaagctggaaatgaagccaaagtacccgcactgcgaggagaagatggttatcatcaccaccaagagcgtgtccaggtaccgaggtcaggagcactgcctgcaccccaagctgcagagcaccaagcgcttcatcaagtggtacaacgcctggaacgagaagcgcagggtctacgaagaatagggtgaaaaacctcagaagggaaaactccaaaccagttgggagacttgtgcaaaggactttgcagattaaaaaaaaaaaaaaaaaaaaaaaaaaaaaaaaaaaaaaaaaaagcctttctttctcacaggcataagacacaaattatatattgttatgaagcactttttaccaacggtcagtttttacattttatagctgcgtgcgaaaggcttccagatgggagacccatctctcttgtgctccagacttcatcacaggctgctttttatcaaaaaggggaaaactcatgcctttcctttttaaaaaatgcttttttgtatttgtccatacgtcactatacatctgagctttataagcgcccgggaggaacaatgagcttggtggacacatttcattgcagtgttgctccattcctagcttgggaagcttccgcttagaggtcctggcgcctcggcacagctgccacgggctctcctgggcttatggccggtcacagcctcagtgtgactccacagtggcccctgtagccgggcaagcaggagcaggtctctctgcatctgttctctgaggaactcaagtttggttgccagaaaaatgtgcttcattcccccctggttaatttttacacaccctaggaaacatttccaagatcctgtgatggcgagacaaatgatccttaaagaaggtgtggggtctttcccaacctgaggatttctgaaaggttcacaggttcaatatttaatgcttcagaagcatgtgaggttcccaacactgtcagcaaaaaccttaggagaaaacttaaaaatatatgaatacatgcgcaatacacagctacagacacacattctgttgacaagggaaaaccttcaaagcatgtttctttccctcaccacaacagaacatgcagtactaaagcaatatatttgtgattccccatgtaattcttcaatgttaaacagtgcagtcctctttcgaaagctaagatgaccatgcgccctttcctctgtacatatacccttaagaacgccccctccacacactgccccccagtatatgccgcattgtactgctgtgttatatgctatgtacatgtcagaaaccattagcattgcatgcaggtttcatattctttctaagatggaaagtaataaaatatatttgaaatgtaccaaaa

SEQ ID NO: 21 - protein, 97 aa, Homo sapiens
MKVSAALLWLLLIAAAFSPQGLAGPASVPTTCCFNLANRKIPLQRLESYRRITSGKCPQKAVIFKTKLAKDICADPKKKWVQDSMKYLDQKSPTPKP

SEQ ID NO: 22 - DNA, 1005 bp, Homo sapiens
agagaggctgagaccaacccagaaaccaccacctctcacgccaaagctcacaccttcagcctccaacatgaaggtctccgcagcacttctgtggctgctgctcatagcagctgccttcagcccccaggggctcgctgggccagcttctgtcccaaccacctgctgctttaacctggccaataggaagataccccttcagcgactagagagctacaggagaatcaccagtggcaaatgtccccagaaagctgtgatcttcaagaccaaactggccaaggatatctgtgccgaccccaagaagaagtgggtgcaggattccatgaagtatctggaccaaaaatctccaactccaaagccataaataatcaccatttttgaaaccaaaccagagcctgagtgttgcctaatttgttttcccttcttacaatgcattctgaggtaacctcattatcagtccaaagggcatgggttttattatatatatatatttttttttttaaaaaaaaaacgtattgcatttaatttattgaggctttaaaacttatcctccatgaatatcagttatttttaaactgtaaagctttgtgcagattctttaccccctgggagccccaattcgatcccctgtcacgtgtgggcaatgttccccctctcctctcttcctccctggaatcttgtaaaggtcctggcaaagatgatcagtatgaaaatgtcattgttcttgtgaacccaaagtgtgactcattaaatggaagtaaatgttgttttaggaatacataaagtatgtgcatattttattatagtcactagttgtaatttttttgtgggaaatccacactgagctgagggggacaaagatggctgtggccaagaggggcttggttaagggggtgggaactatgtccctgggaaatgagtttttggcttagctggtcttcattgaaatgcagggtgaaactgacaaacccattccagccctctattcccattttcaacagtatttcc

SEQ ID NO: 23 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 24 - DNA, 1175 bp, Homo sapiens
gagacattcctcaattgcttagacatattctgagcctacagcagaggaacctccagtctcagcaccatgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttaaaaccagaggggagcaaaatcgatgcagtgcttccaaggatggaccacacagaggctgcctctcccatcacttccctacatggagtatatgtcaagccataattgttcttagtttgcagttacactaaaaggtgaccaatgatggtcaccaaatcagctgctactactcctgtaggaaggttaatgttcatcatcctaagctattcagtaataactctaccctggcactataatgtaagctctactgaggtgctatgttcttagtggatgttctgaccctgcttcaaatatttccctcacctttcccatcttccaagggtactaaggaatctttctgctttggggtttatcagaattctcagaatctcaaataactaaaaggtatgcaatcaaatctgctttttaaagaatgctctttacttcatggacttccactgccatcctcccaaggggcccaaattctttcagtggctacctacatacaattccaaacacatacaggaaggtagaaatatctgaaaatgtatgtgtaagtattcttatttaatgaaagactgtacaaagtagaagtcttagatgtatatatttcctatattgttttcagtgtacatggaataacatgtaattaagtactatgtatcaatgagtaacaggaaaattttaaaaatacagatagatatatgctctgcatgttacataagataaatgtgctgaatggttttcaaaataaaaatgaggtactctcctggaaatattaagaaagactatctaaatgttgaaagatcaaaaggttaataaagtaattataactaa

SEQ ID NO: 25 - protein, 241 aa, Homo sapiens
MPRGFTWLRYLGIFLGVALGNEPLEMWPLTQNEECTVTGFLRDKLQYRSRLQYMKHYFPINYKISVPYEGVFRIANVTRLRAQVSERELRYLWVLVSLSATESVQDVLLEGHPSWKYLQEVETLLLNVQQGLTDVEVSPKVESVLSLLNAPGPNLKLVRPKALLDNCFRVMELLYCSCCKQSSVLNWQDCEVPSPQSCSPEPSLQYAATQLYPPPPWSPSSPPHSTGSVRPVRAQGEGLLP

SEQ ID NO: 26 - DNA, 1772 bp, Homo sapiens
agacgggaagcctggactgtgggttgggggcagcctcagcctctccaacctggcacccactgcccgtggcccttaggcacctgcttggggtcctggagccccttaaggccaccagcaaatcctaggagaccgagtcttggcacgtgaacagagccagatttcacactgagcagctgcagtcggagaaatcagagaaagcgtcacccagccccagattccgaggggcctgccagggactctctcctcctgctccttggaaaggaagaccccgaaagacccccaagccaccggctcagacctgcttctgggctgccatgggacttgcggccaccgccccccggctgtcctccacgctgccgggcagataagggcagctgctgcccttggggcacctgctcactcccgcagcccagccactcctccagggccagcccttccctgactgagtgaccacctctgctgccccgaggccatgtaggccgtgcttaggcctctgtggacacactgctggggacggcgcctgagctctcagggggacgaggaacaccaccatgccccggggcttcacctggctgcgctatcttgggatcttccttggcgtggccttggggaatgagcctttggagatgtggcccttgacgcagaatgaggagtgcactgtcacgggttttctgcgggacaagctgcagtacaggagccgacttcagtacatgaaacactacttccccatcaactacaagatcagtgtgccttacgagggggtgttcagaatcgccaacgtcaccaggctgagggcccaggtgagcgagcgggagctgcggtatctgtgggtcttggtgagcctcagtgccactgagtcggtgcaggacgtgctgctcgagggccacccatcctggaagtacctgcaggaggtggagacgctgctgctgaatgtccagcagggcctcacggatgtggaggtcagccccaaggtggaatccgtgttgtccctcttgaatgccccagggccaaacctgaagctggtgcggcccaaagccctgctggacaactgcttccgggtcatggagctgctgtactgctcctgctgtaaacaaagctccgtcctaaactggcaggactgtgaggtgccaagtcctcagtcttgcagcccagagccctcattgcagtatgcggccacccagctgtaccctccgcccccgtggtcccccagctccccgcctcactccacgggctcggtgaggccggtcagggcacagggcgagggcctcttgccctgagcaccctggatggtgactgcggataggggcagccagaccagctcccacaggagttcaactgggtctgagacttcaaggggtggtggtgggagccccccttgggagaggacccctgggaagggtgtttttcctttgagggggattctgtgccacagcagggctcagcttcctgccttccatagctgtcatggcctcacctggagcggaggggacctggggacctgaaggtggatggggacacagctcctggcttctcctggtgctgccctcactgtccccccgcctaaagggggtactgagcctcctgtggcccgcagcagtgagggcacagctgtgggttgcaggggagacagccagcacggcgtggccattctatgaccccccagcctggcagactggggagctgggggcagagggcggtgccaagtgccacatcttgccatagtggatgctcttccagtttcttttttctattaaacaccccacttcctttgg

SEQ ID NO: 27 - protein, 693 aa, Homo sapiens
MKENYCLQAALVCLGMLCHSHAFAPERRGHLRPSFHGHHEKGKEGQVLQRSKRGWVWNQFFVIEEYTGPDPVLVGRLHSDIDSGDGNIKYILSGEGAGTIFVIDDKSGNIHATKTLDREERAQYTLMAQAVDRDTNRPLEPPSEFIVKVQDINDNPPEFLHETYHANVPERSNVGTSVIQVTASDADDPTYGNSAKLVYSILEGQPYFSVEAQTGIIRTALPNMDREAKEEYHVVIQAKDMGGHMGGLSGTTKVTITLTDVNDNPPKFPQSVYQMSVSEAAVPGEEVGRVKAKDPDIGENGLVTYNIVDGDGMESFEITTDYETQEGVIKLKKPVDFETKRAYSLKVEAANVHIDPKFISNGPFKDTVTVKISVEDADEPPMFLAPSYIHEVQENAAAGTVVGRVHAKDPDAANSPIRYSIDRHTDLDRFFTINPEDGFIKTTKPLDREETAWLNITVFAAEIHNRHQEAKVPVAIRVLDVNDNAPKFAAPYEGFICESDQTKPLSNQPIVTISADDKDDTANGPRFIFSLPPEIIHNPNFTVRDNRDNTAGVYARRGGFSRQKQDLYLLPIVISDGGIPPMSSTNTLTIKVCGCDVNGALLSCNAEAYILNAGLSTGALIAILACIVILLGCPSLMEPPSPREDMRLLYLGFQLMLFSYVKVNRRFCLLGVFIKLPFLYVVATESPTTLTSL

SEQ ID NO: 28 - DNA, 6901 bp, Homo sapiens
attccgcggccgccgccagctgaggggagcgtcgcgggccgaggagcagatgccgcgggggccgctcgcagccgccgctgacttgtgaatgggaccgggactggggccgggactgacaccgcagcgcttgccctgcgccagggactggcggctcggaggttgcgtccaccctcaagggccccagaaatcactgtgttttcagctcagcggccctgtgacattccttcgtgttgtcatttgttgagtgaccaatcagatgggtggagtgtgttacagaaattggcagcaagtatccaatgggtgaagaagaagctaactggggacgtgggcagccctgacgtgatgagctcaaccagcagagacattccatcccaagagaggtctgcgtgacgcgtccgggaggccaccctcagcaagaccaccgtacagttggtggaaggggtgacagctgcattctcctgtgcctaccacgtaaccaaaaatgaaggagaactactgtttacaagccgccctggtgtgcctgggcatgctgtgccacagccatgcctttgccccagagcggcgggggcacctgcggccctccttccatgggcaccatgagaagggcaaggaggggcaggtgctacagcgctccaagcgtggctgggtctggaaccagttcttcgtgatagaggagtacaccgggcctgaccccgtgcttgtgggcaggcttcattcagatattgactctggtgatgggaacattaaatacattctctcaggggaaggagctggaaccatttttgtgattgatgacaaatcagggaacattcatgccaccaagacgttggatcgagaagagagagcccagtacacgttgatggctcaggcggtggacagggacaccaatcggccactggagccaccgtcggaattcattgtcaaggtccaggacattaatgacaaccctccggagttcctgcacgagacctatcatgccaacgtgcctgagaggtccaatgtgggaacgtcagtaatccaggtgacagcttcagatgcagatgaccccacttatggaaatagcgccaagttagtgtacagtatcctcgaaggacaaccctatttttcggtggaagcacagacaggtatcatcagaacagccctacccaacatggacagggaggccaaggaggagtaccacgtggtgatccaggccaaggacatgggtggacatatgggcggactctcagggacaaccaaagtgacgatcacactgaccgatgtcaatgacaacccaccaaagtttccgcagagcgtataccagatgtctgtgtcagaagcagccgtccctggggaggaagtaggaagagtgaaagctaaagatccagacattggagaaaatggcttagtcacatacaatattgttgatggagatggtatggaatcgtttgaaatcacaacggactatgaaacacaggagggggtgataaagctgaaaaagcctgtagattttgaaaccaaaagagcctatagcttgaaggtagaggcagccaacgtgcacatcgacccgaagtttatcagcaatggccctttcaaggacactgtgaccgtcaagatctcagtagaagatgctgatgagccccctatgttcttggccccaagttacatccacgaagtccaagaaaatgcagctgctggcaccgtggttgggagagtgcatgccaaagaccctgatgctgccaacagcccgataaggtattccatcgatcgtcacactgacctcgacagatttttcactattaatccagaggatggttttattaaaactacaaaacctctggatagagaggaaacagcctggctcaacatcactgtctttgcagcagaaatccacaatcggcatcaggaagccaaagtcccagtggccattagggtccttgatgtcaacgataatgctcccaagtttgctgccccttatgaaggtttcatctgtgagagtgatcagaccaagccactttccaaccagccaattgttacaattagtgcagatgacaaggatgacacggccaatggaccaagatttatcttcagcctaccccctgaaatcattcacaatccaaatttcacagtcagagacaaccgagataacacagcaggcgtgtacgcccggcgtggagggttcagtcggcagaagcaggacttgtaccttctgcccatagtgatcagcgatggcggcatcccgcccatgagtagcaccaacaccctcaccatcaaagtctgcgggtgcgacgtgaacggggcactgctctcctgcaacgcagaggcctacattctgaacgccggcctgagcacaggcgccctgatcgccatcctcgcctgcatcgtcattctcctgggttgcccaagcttaatggaacccccctctcccagggaagacatgagattgctttatctgggcttccagctgatgctattttcctatgttaaagtaaacagaagattttgtcttctgggggtctttataaaacttcctttcctctatgtggtggctacagagagtccaaccacacttacgtcattgtagtattgtttgtgaccctgagaaggcaaaagaaagaaccactcattgtctttgaggaagaagatgtccgtgagaacatcattacttatgatgatgaagggggtggggaagaagacacagaagcctttgatattgccaccctccagaatcctgatggtatcaatggatttatcccccgcaaagacatcaaacctgagtatcagtacatgcctagacctgggctccggccagcgcccaacagcgtggatgtcgatgacttcatcaacacgagaatacaggaggcagacaatgaccccacggctcctccttatgactccattcaaatctacggttatgaaggcaggggctcagtggccgggtccctgagctccctagagtcggccaccacagattcagacttggactatgattatctacagaactggggacctcgttttaagaaactagcagatttgtatggttccaaagacacttttgatgacgattcttaacaataacgatacaaatttggccttaagaactgtgtctggcgttctcaagaatctagaagatgtgtaaacaggtatttttttaaatcaaggaaaggctcatttaaaacaggcaaagttttacagagaggatacatttaataaaactgcgaggacatcaaagtggtaaatactgtgaaataccttttctcacaaaaaggcaaatattgaagttgtttatcaacttcgctagaaaaaaaaaacacttggcatacaaaatatttaagtgaaggagaagtctaacgctgaactgacaatgaagggaaattgtttatgtgttatgaacatccaagtctttcttcttttttaagttgtcaaagaagcttccacaaaattagaaaggacaacagttctgagctgtaatttcgccttaaactctggacactctatatgtagtgcatttttaaacttgaaatatataatattcagccagcttaaacccatacaatgtatgtacaatacaatgtacaattatgtctcttgagcatcaatcttgttactgctgattcttgtaaatctttttgcttctactttcatcttaaactaatacgtgccagatataactgtcttgtttcagtgagagacgccctatttctatgtcatttttaatgtatctatttgtacaattttaaagttcttattttagtatacgtataaatatcagtattctgacatgtaagaaaatgttacggcatcacacttatattttatgaacattgtactgttgctttaatatgagcttcaatataagaagcaatctttgaaataaaaaaagatttttttttaattctgggtttgattcttaacattgaaacaaacgttaagtatttctaatgatccatttatatttctaatttaattgtgatcttttaataaccctatttatgatctgttgttgtctgtctgctgcttttattgtttatttaaaatcaaatatgttttacaaatgttttttcagacaagattctgtaacatcatgtaaagcttttttgtacattcttggtgttaacctcctggcttctcttcacacacatcttctaaaaaagaaggatgtgaaagaactaggtcagtctatgactttgcaatatgtgttatatagtatgcatttatcttgtatatcagtaatttgatggttatgagagatgaatccatgagggaatggagctatcagaactctaatgttccaggtatacattctatgccccacactgagcactggggaactgggggactagagtcaaaaatataaatttgcccagactctaatgttattctattttttcttctgttgaacttaccaggctattgtaagactcttgatagttgaaactgcttatttttcctcctgtaattttaactaattgtaaaatgatgtggcattttatgttttaatgagaatgggcgattcatttaaaaaagctttgtttagaatatgcttggggccgtaagctcagaatgagggcagggaccattttggattctgagagtcgatgccatttggtccaggagtgtgtctacagtcccctgcattccagctagtttcttggggattgaaacttatgtgaagggcatttcacctgttcagttgggccaaaggtcaaaacgtagcaatacttggggaaagaccacataaagtcacactgcaagtgctttccctctttccccctacacacagggcacgtgctttttcttggattgcagacaatttttacagtttttttctgactttattgtgaaagtttgtttcaagcatttcttgatatcatgttatgtactatttttatgatttagtcaacatgcatacaaagaaatgttttttatgaagtgctcacttccattttactttgcattgaaatcaaattgggctgaacacttcaatggaatacattttgtggacaatgtcactttagaatctttcatctcagtgaaggattacacattctcaatacttccataattgcaggttgtgttcatttttttatatagtttttgtaatccaaagaatattttgctagatttgcacagatctccaattgaatttgcaatgaagaaataactcaaaaggaatatgaatagcatttaaataagtatacagctgtaagtaaccctgtcaccatggatgatccttttctctaggaatgtatttggattagagatgacaactacattttcgcatttttatgttgaagtcttttttaaaaaggctgtttacttttcagtagttaagaatacttgtttttctttttcttttttttttttttttttaccttttattttttcgttaagcctctattgtttgtagaacactcttagaaacttggaaataaaatgtctttcccaactagtggagtcctttttcatttggagcacattgccttaaaagaagtcttaatttaaacggtccttccttattctaaagtaatcactgttttataccatctatgcagctaaaagaaggaacatgcttctgttcttttcctcaagtaatggttattgtttctagtcatcattcattcattgattcattcattaattcatcaaaatcttattttataaaccctgttccacttactggaggattcagaatgaatcttactaccttttctgacatcttttgataattcagccctgtaccaaagtatccaccttgttgtcttataatcacctatttacctatttgccctcctagaaaatgcaagaagatattttctctccttccaaattgaaggaagaacataaaagatataacaggaaggagatggtgagatatagagtgtgagcggaaattaggccagctgtggcaattctggacagatcttgggtttagctaagttatttcttttaggcctgggtttctgggggtgacagggaagataaaagagtagtttatttgcacctcttggagaattgcttaaaaatatagagatcatggctctgtatgtcaggtggaaccaggtcaggagtatttgaaactgctcctgggtcattgtgacatatccttcacatctttttgagaaactttataagacaatgggggtgaatgggggctgggcagttggagtctctgagcagaagaggggcaaaatttatttggcaggcagtgtggaggacagattaggagcatataaacccagaggtgtgccccaggagggcttttgcaaaggtcaatatgagatagaatgagggcctgaaataattcagtaatttggagatggagaagaggaaagacttctctgctcttgcactgccatcagcctggtctgggccatggtcatctctgacccggaagactgaccccacctcttggctcaccctctgcctcccaacctcctcttcacaaagaagccagagggatacttttaacacacaacccagatcacatgacttcgtaacttaaacctcttcactggcttcccaaagacttaaaatgaattctgatgcctttattttattgctttacatgaacagggccctgcgaacctctccagtgtcattccactccatcctcctttcagtgcacgatgctccagccacactggccatctttcggttcctgatacaaaaaaaaacacgttccttttccatggaaagcaggtcacccttgttattttgtatcgatgacaactctttaaacttattttgctttttggctttatgtatgtgtgtgggtgggtgggactgactgccccactagaatgtaagctccatgagggcagggaatcttgctttcttgtttaccattgtatactcagttctttacacagtgcctgaaacataacaggtacacaataaatatctattgaatgaaagcaaa

(19) United States
(12) Patent Application Publication
(10) Pub. No.: US 2022/0143099 A1
(43) Pub. Date: May 12, 2022

(54) METHODS TO ENHANCE T CELL REGENERATION

(71) Applicants: The General Hospital Corporation, Boston, MA (US); President and Fellows of Harvard College, Cambridge, MA (US)

(72) Inventors: David T. SCADDEN, Boston, MA (US); Karin GUSTAFSSON, Boston, MA (US)

(21) Appl. No.: 17/598,963

(22) PCT Filed: Apr. 1, 2020
(86) PCT No.: PCT/US2020/026170

§ 371 Date: Sep. 28, 2021

Related U.S. Application Data
(60) Provisional application No. 62/945,290, filed on Dec. 9, 2019.
(60) Provisional application No. 62/828,384, filed on Apr. 2, 2019.

(51) Int. Cl. A61K 35/28; C12N 5/0775; A61K 38/17; A61K 38/19; A61K 38/20; A61P 37/04; A61K 38/18

(52) U.S. Cl. CPC A61K 35/28; C12N 5/0668; A61K 38/1709; A61K 38/179; A61K 38/195; A61K 38/1875; A61K 38/2086; A61P 37/04; A61K 38/20; A61K 38/208; A61K 38/193

(57) ABSTRACT

Described herein are methods for the restoration of T cell production in a subject in need thereof.

### CROSS-REFERENCE TO RELATED APPLICATION(S)

This application relates to and claims priority from U.S. Patent Application No. 62/828,384 filed on Apr. 2, 2019 and from U.S. Patent Application No. 62/945,290 filed on Dec. 9, 2019, the entire disclosure of which is incorporated herein by reference.

### STATEMENT OF RIGHTS TO INVENTIONS MADE UNDER FEDERALLY SPONSORED RESEARCH

This work was supported by a National Institutes of Health Grant No. DK107784. The government may have certain rights to the invention.

### SEQUENCE LISTING

The instant application contains a sequence listing which has been submitted electronically in ASCII format and is hereby incorporated by reference in its entirety. The ASCII copy, created on Mar. 31, 2020, is named 51395-002WO3_Sequence_Listing_03.31.20_ST25 and is 87,125 bytes in size.

### BACKGROUND OF THE INVENTION

T cell deficiency is an acute and lethal complication of hematopoietic stem cell transplantation (HSCT) and is a common, progressive feature of aging. Generation of new T cells depends on hematopoietic stem/progenitor cells entering and maturing in the thymus. Methods to enhance thymic tissue regeneration and long-term T cell reconstitution would be highly desirable.

### SUMMARY OF THE INVENTION

In one aspect, the invention provides a method for increasing the production of T cells within a T-cell producing tissue or fluid of a subject in need thereof, said method comprising administering a composition comprising mesenchymal stromal cells into a T-cell producing tissue or fluid of the subject, wherein the mesenchymal stromal cells express Periostin and Pdgfra, thereby increasing the production of T cells within the T-cell producing tissue or fluid of the subject.

In one embodiment, the mesenchymal stromal cells do not express Cdh11 and CD248.

In another embodiment, the T-cell producing tissue is thymus.

In another embodiment, the T-cell producing tissue is a lymphopoietic tissue.

In yet another embodiment, the T-cell producing fluid is blood.

In yet another embodiment, the subject has undergone hematopoietic stem cell transplantation.

In yet another embodiment, the subject has one or more of a condition associated with T lymphopenia, a T cell production disorder, a T cell function disorder, a distorted repertoire of T cell receptor bearing cells, an infection or a tumor.

In yet another embodiment, the mesenchymal stromal cells express Flt3 ligand (fins related receptor tyrosine kinase 3 ligand), Ccl19 (C-C motif chemokine ligand 19), BMP2 (bone morphogenetic protein 2), BMP4 (bone morphogenetic protein 4), IL-15 (interleukin 15), IL-12a (interleukin-12a), Cxcl14 (C-X-C motif chemokine ligand 14), Ccl11 (C-C motif chemokine ligand 11), (Cxcl10 C-X-C motif chemokine ligand 10), or IL-34 (interleukin 34) and combinations thereof.

In one embodiment, the mesenchymal stromal cells express Ccl19, Flt31, and IL-15.

In yet another embodiment, the mesenchymal stromal cells express Flt3 ligand, Ccl19, IL-15 and do not express Cdh11 and CD248.

In yet another embodiment, the mesenchymal stromal cells are autologous to the subject.

In yet another embodiment, the mesenchymal stromal cells are derived from mesenchymal stem cells or progenitors thereof.

In yet another embodiment, the mesenchymal stromal cells are derived from embryonic stem cells or progenitors thereof.

In yet another embodiment, the mesenchymal stromal cells are derived from iPS cells or progenitors thereof.

In another aspect, the invention provides a method for increasing the production of T cells within a T-cell producing tissue or fluid of a subject in need thereof, said method comprising administering a composition comprising Ccl19 (C-C motif chemokine ligand 19) into a T-cell producing tissue or fluid of the subject, thereby increasing the production of T cells within the T-cell producing tissue or fluid of the subject.

In one embodiment, the T-cell producing tissue is thymus.

In another embodiment, the T-cell producing tissue is a lymphopoietic tissue.

In yet another embodiment, the T-cell producing fluid is blood.

In yet another embodiment, the subject has undergone hematopoietic stem cell transplantation.

In yet another embodiment, the subject has one or more of a condition associated with T lymphopenia, a T cell production disorder, a T cell function disorder, a distorted repertoire of T cell receptor bearing cells, an infection or a tumor.

In yet another aspect, the invention provides isolated mesenchymal stromal cells expressing Periostin and Pdgfra.

In one embodiment, the mesenchymal stromal cells do not express Cdh11 and CD248.

In another embodiment, the mesenchymal stromal cells express Flt3 ligand (fms related receptor tyrosine kinase 3 ligand), Ccl19 (C-C motif chemokine ligand 19), BMP2 (bone morphogenetic protein 2), BMP4 (bone morphogenetic protein 4), IL-15 (interleukin 15), IL-12a (interleukin-12a), Cxcl14 (C-X-C motif chemokine ligand 14), Ccl11 (C-C motif chemokine ligand 11), (Cxcl10 C-X-C motif chemokine ligand 10), or IL-34 (interleukin 34,) and combinations thereof.

In yet another embodiment, the mesenchymal stromal cells express Ccl19, Flt31, and IL-15.

In yet another embodiment, the mesenchymal stromal cells express Ccl19, Flt3 ligand and IL-15, and do not express Cdh11 and CD248.

In yet another embodiment, the mesenchymal stromal cells are derived from mesenchymal stem cells or progenitors thereof.

In yet another embodiment, the mesenchymal stromal cells are derived from embryonic stem cells or progenitors thereof.

In yet another embodiment, the mesenchymal stromal cells are derived from iPS cells or progenitors thereof.

In yet another aspect, the invention provides a population of isolated stem cells capable of differentiating into mesenchymal stromal cells, wherein said mesenchymal stromal cells express Periostin and Pdgfra.

In one embodiment, the mesenchymal stromal cells do not express Cdh11 and CD248.

In yet another aspect, the invention provides a composition for increasing the production of T cells within a T-cell producing tissue or fluid of a subject, said composition comprising Ccl19 (C-C motif chemokine ligand 19).

Other features and advantages of the invention will be apparent from the Detailed Description, and from the claims. Thus, other aspects of the invention are described in the following disclosure and are within the ambit of the invention.

### BRIEF DESCRIPTION OF THE DRAWINGS

The following Detailed Description, given by way of example, but not intended to limit the invention to specific embodiments described, may be understood in conjunction with the accompanying figures, incorporated herein by reference.

FIG. 1 shows that thymus MSCs express key lymphopoietic factors. (A) Study overview human thymus samples. (B) tSNE showing annotation of major thymus stromal cell types in human. (C) Number of cells in each population in human thymus as determined by scRNAseq and flow cytometry. (D) Expression of key lymphopoietic regulators within the stromal compartment in human thymus shown as a heatmap. (E) Study overview murine samples (F) tSNE showing annotation of major thymus stromal cell types in mouse. (G) Number of cells in each population in murine thymus as determined by scRNAseq and flow cytometry. (H) Expression of key lymphopoietic regulators within the stromal compartment in human thymus shown as a heatmap. (I) Quantification of Il15, Ccl19, Flt31 and Bmp4 expression across all thymus stromal cell types in murine samples.

FIG. 2 depicts (A) Gating strategy for flow cytometric isolation of human thymus stromal cells. (B) Comparisons of stromal yield using two different digestion protocols for human thymus processing. (C) tSNE displaying all sequenced cells from human samples, including hematopoietic cells. (D) Definition of human hematopoietic cells based on key marker genes. (E) tSNE showing the annotation of major thymus stromal cell clusters in human samples. (F) Gating strategy for flow validation of the major thymus stromal cell clusters in humans. (G) Gating strategy for flow cytometric isolation of mouse thymus stromal cells (H) Number of UMIs and genes per cell in mouse samples (I) tSNE displaying all sequenced cells from mouse samples, including hematopoietic cells. (J) The major steps of T cell development can be traced through the expression of key marker genes. (K) tSNE showing the annotation of major thymus stromal cell clusters in murine samples. (L) Heat map displaying the top differentially expressed genes among murine thymus stromal cells. (M) Gating strategy for flow validation of the major thymus stromal cell clusters in humans.

FIG. 3 depicts (A) tSNE showing three subsets of thymic MSCs in human and mouse thymus. (B) GO term analysis of significantly differentially expressed genes in different murine MSC subsets. (C) Expression of C119, Flt31 and IL15 in human and murine MSC subsets.

FIG. 4 depicts (A) Heat map displaying the top differentially expressed genes among murine thymus MSCs. (B) Expression of marker genes defining human and murine MSC subsets. (C) Quantification of thymus MSC subsets in human and murine samples. (D) tSNE displaying all sequenced stromal cells from Bornstein et. al. (E) tSNE showing three subsets of thymic MSCs. (F) Expression of MSC subset marker genes in Bornstein et. al. data set. (G) GO term analysis of significantly differentially expressed genes in murine CD248 MSCs.

FIG. 5 depicts the loss of Periostin+ MSCs following radiation conditioning. (A) Experiment overview. (B) Two-photon microscopy image showing GFP labeled cells arriving in the tissue 3 days post-transplantation, 4 days post-irradiation. (C) tSNEs displaying thymus stromal cells from non-treated control mice (Control) and irradiated and transplanted recipient mice (Transplantation). (D) Compositional changes in the thymus MSC compartment following irradiation and transplantation. (E) GO term analysis of thymus MSC populations after irradiation and transplantation.

FIG. 6 depicts (A) Experiment overview. (B) Quantification of GFP labeled cells arriving in the tissue by flow cytometry. (C) Two-photon microscopy image showing the thymus after irradiation and transplantation. (D) Two-photon microscopy image showing the absence or presence of GFP+ cells in the tissue at 2, 4 and 5 days post-transplantation. (E) Compositional changes in the thymus stroma compartment following irradiation and transplantation. (F) Changes in expression of secreted factors, Flt31, Cc119 and IL15 in MSC subsets following irradiation and transplantation.

FIG. 7 depicts transfer of thymus CD248-MSCs accelerates T cell production following radiation conditioning. (A) Experiment overview. (B) Flow validation of thymus regeneration 6 days bone marrow transplantation and intrathymic transfer of CD248-MSCs. (C) Flow validation of the effect of MSC GFP and Ccl19 knockout on thymus regeneration 6 days bone marrow transplantation and intrathymic transfer of MSCs. (D) Flow validation and sjTREC measurement in the thymus 1 month bone marrow transplantation and intrathymic transfer of MSCs to determine rate of de novo T cell generation. (E) 16 weeks follow-up of T cell recovery following bone marrow transplantation and intrathymic transfer of MSCs. (F) Estimation of vaccination response 54 days following bone marrow transplantation and intrathymic transfer of MSCs demonstrates functionality of the newly generated T cells.

FIG. 8 depicts (A) Establishment of CD9912 and Itgb5 as pan-MSC markers for flow cytometric isolation. (B) Colony forming ability of CD9912+ Itgb5+ thymus MSCs. (C) Validation of Pdgfra and CD248 as flow cytometric markers to distinguish between MSC subsets. (D) Analysis of different T cell developmental steps 1 month bone marrow transplantation and intrathymic transfer of MSCs. (E) 16 weeks follow-up of B cell and myeloid cell recovery following bone marrow transplantation and intrathymic transfer of MSCs. (F) Flow validation of presence of GFP labeled MSCs in the thymus of recipient mice 16 weeks post-transfer.

FIG. 9 depicts Periostin+ MSCs specifically enhancing T cell progenitor recruitment. (A) Gating strategy for flow cytometric isolation of thymic tdTomato+ (Penk+) MSCs and tdTomato− (Postn+) MSCs. (B) Experiment overview. (C) Flow validation of thymus regeneration 6 days bone marrow transplantation and intrathymic transfer of tdTomato+ (Penk+) MSCs and tdTomato− (Postn+) MSCs.

### DETAILED DESCRIPTION OF THE INVENTION

### Definitions

Unless otherwise defined, all technical and scientific terms used herein have the same meaning as commonly understood by one of ordinary skill in the art to which this invention belongs. In case of conflict, the present application, including definitions will control.

A “subject” is a vertebrate, including any member of the class mammalia, including humans, domestic and farm animals, and zoo, sports or pet animals, such as mouse, rabbit, pig, sheep, goat, cattle and higher primates.

As used herein, the terms “treat,” “treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

By “effective amount” is meant the amount of mesenchymal cells, stem cells or progenitor cells that produce the desired therapeutic response (i.e., enhancing T cell production in the thymus).

By “mesenchymal progenitor cell” is meant a multipotent cell which has the potential to become committed to the mesenchymal lineage.

By “mesenchymal stem cell” is meant a pluripotent cell which has the potential to become committed to multiple mesenchymal cell types but does not express genes defining a specific cell type.

By “isolated” is meant a material that is free to varying degrees from components which normally accompany it as found in its native state. “Isolate” denotes a degree of separation from original source or surroundings.

As used herein “an increase” refers to an amount of T-cell production that is at least about 0.05 fold more (for example 0.1, 0.2, 0.3, 0.4, 0.5, 1, 5, 10, 25, 50, 100, 1000, 10,000-fold or more) than the amount of T-cell production compared to a reference level (e.g., a subject having normal T-cell production). “Increased” as it refers to an amount of T-cell production also means at least about 5% more (for example 5, 6, 7, 8, 9, 10, 15, 20, 25, 30, 35, 40, 45, 50, 55, 60, 65, 70, 75, 80, 85, 90, 95, 99 or 100% more) than the amount of T-cell production compared to a reference level (e.g., a subject having normal T-cell production). Amounts can be measured according to methods known in the art for determining amounts of T-cells.

Unless specifically stated or clear from context, as used herein, the term “about” is understood as within a range of normal tolerance in the art, for example within 2 standard deviations of the mean. “About” is understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

Other definitions appear in context throughout this disclosure.

### Compositions and Methods of the Invention

Comprehensive analysis of mesenchymal stromal cells derived from the thymus identified a Periostin positive, Pdgfra positive immunophenotype (Periostin+Pdgfra+ immunophenotype) that has now been determined to be critical for T cell production. Adoptive cell transfer of these subpopulations of cells into a T cell producing tissue or fluid, such as the thymus, has shown that these cells are capable of enhancing thymic tissue regeneration and long-term T cell reconstitution in the context of Hematopoietic Stem Cell Transplant (HSCT).

Generation or isolation of and transfer of Periostin+Pdgfra+ cells, and/or specific genes or proteins they express, provides a therapeutic benefit in the setting of HSCT or other circumstances where T cell depletion/deficiency or dysfunction contributes to adverse effects including advanced age.

Periostin is described, for example, by GenBank Accession No. NM_001135934.2 (SEQ ID NOs: 1 and 2). Periostin, also called osteoblast-specific factor 2, is a secreted cell adhesion protein, which shares a homology with the insect cell adhesion molecule fasciclin I. Its N-terminal region contains a signal peptide (SP) for its secretion, and a cysteine-rich region (EMI domain) which promotes the formation of multimers in non-reducing conditions. Adjacent to the SP and the EMI domains, four internal homologous repeats (FAS domains) are located; these are homologous to the insect cell adhesion protein fasciclin I and act as ligands for the integrins. The C-terminal region of periostin consists of a hydrophilic domain. The N-terminal region of periostin is highly conserved, while the C-terminal region of the protein varies depending on the isoform. The N-terminal region regulates the cell function by binding to integrins at the plasma membrane of the cells through its FAS domains. The C-terminal region of the protein regulates the cell-matrix organization and interactions by binding extracellular matrix (ECM) proteins such as collagen I/V, fibronectin, tenascin C, acid mucopolysaccharides, such as heparin and periostin itself.

Periostin has been shown to be an important regulator of bone and tooth formation and maintenance, and of cardiac development and healing. Periostin also plays an important role in tumor development and is upregulated in a wide variety of cancers such as colon, pancreatic, ovarian, breast, head and neck, thyroid, and gastric cancer as well as in neuroblastoma. Periostin binding to the integrins activates the Akt/PKB- and FAK-mediated signaling pathways which lead to increased cell survival, angiogenesis, invasion, metastasis, and importantly, epithelial-mesenchymal transition of carcinoma cells.

Platelet Derived Growth Factor Receptor Alpha or Pdgfra is a cell surface tyrosine kinase receptor for members of the platelet-derived growth factor family. These growth factors are mitogens for cells of mesenchymal origin. Pdgfra is known to play a role in organ development, wound healing, and tumor progression. Pdgfra is described, for example, by GenBank Accession NM_001347827.2 (SEQ ID NOs: 3 and 4). Pdgfra is a typical receptor tyrosine kinase, which is a transmembrane protein consisting of an extracellular ligand binding domain, a transmembrane domain and an intracellular tyrosine kinase domain. The molecular mass of the mature, glycosylated PDGFRα protein is approximately 170 kDA.

Periostin+Pdgfra+ Mesenchymal stromal cells identified by the Periostin+Pdgfra+ immunophenotype differentially express genes which promote the regeneration phenotype including, but not limited to Flt3 ligand (fins related receptor tyrosine kinase 3 ligand), Ccl19 (C-C motif chemokine ligand 19), BMP2 (bone morphogenetic protein 2), BMP4 (bone morphogenetic protein 4), IL-15 (interleukin 15), IL-12a (interleukin-12a), Cxcl14 (C-X-C motif chemokine ligand 14), Ccl11 (C-C motif chemokine ligand 11), Cxcl10 (C-X-C motif chemokine ligand 10), and IL-34 (interleukin 34) and combinations thereof. Exemplary combinations include Ccl19, Flt31, and IL-15.

Flt3 ligand is described, for example, by GenBank Accession NM_001204502.2 (SEQ ID NOs: 7 and 8); Ccl19 is described, for example, by GenBank Accession NM_006274.3 (SEQ ID NOs: 9 and 10); BMP2 is described, for example, by GenBank Accession NM_001200.4 (SEQ ID NOs: 11 and 12); BMP4 is described, for example, by GenBank Accession NM_001202.6 (SEQ ID NOs: 13 and 14); IL-15 is described, for example, by GenBank Accession NM_000585.5 (SEQ ID NOs: 15 and 16); IL-12a is described, for example, by GenBank Accession NM_000882.4 (SEQ ID NOs: 17 and 18); Cxcl14 is described, for example, by GenBank Accession NM_004887.5 (SEQ ID NOs: 19 and 20); Ccl11 is described, for example, by GenBank Accession NM_002986.3 (SEQ ID NOs: 21 and 22); Cxcl10 is described, for example, by GenBank Accession NM_001565.4 (SEQ ID NOs: 23 and 24); and IL-34 is described, for example, by GenBank Accession NM_001172771.2 (SEQ ID NOs: 25 and 26). Mesenchymal stromal cells of the invention, or precursors thereof, can be engineered to express or over express these and other regenerative proteins at levels suitable for inducing T cell production.

In some embodiments, mesenchymal stromal cells identified by the Periostin+Pdgfra+ immunophenotype do not express Cdh11 and/or CD248.

Cdh11 gene encodes a type II classical cadherin from the cadherin superfamily, integral membrane proteins that mediate calcium-dependent cell-cell adhesion. Cdh11 is described, for example, by GenBank Accession No. NM_001308392.2 (SEQ ID NOs 27 and 28). Mature cadherin proteins are composed of a large N-terminal extracellular domain, a single membrane-spanning domain, and a small, highly conserved C-terminal cytoplasmic domain. Type II (atypical) cadherins are defined based on their lack of a HAV cell adhesion recognition sequence specific to type I cadherins. Expression of this particular cadherin in osteoblastic cell lines, and its upregulation during differentiation, suggests a specific function in bone development and maintenance.

CD248 is also known as tumor endothelial marker 1, tem1, and endosialin. CD248 is described, for example, by GenBank Accession No. NM_020404.3 (SEQ ID NOs 5 and 6). CD248 is a transmembrane receptor whose known ligands are fibronectin and type I/IV collagen. It is widely expressed on mesenchymal cells during embryonic life and is required for proliferation and migration of pericytes and fibroblasts.

Mesenchymal stromal cells of the invention can be obtained from human tissue (e.g., thymus) according to their Periostin+Pdgfra+ immunophenotype using methods known in the art. Cell purification and isolation methods are known to those skilled in the art and include, but are not limited to, sorting techniques based on cell-surface marker expression, such as fluorescence activated cell sorting (FACS sorting), positive isolation techniques, and negative isolation, magnetic isolation, and combinations thereof. Those skilled in the art can readily determine the percentage of stromal cells, stem cells or their progenitors in a population using various well-known methods, such as FACS. In several embodiments, it will be desirable to first purify the cells. Stromal cells, stem cells or their progenitors may comprise a population of cells that have about 50-55%, 55-60%, 60-65% and 65-70% purity (e.g., non-stromal, non-stem and/or non-progenitor cells have been removed or are otherwise absent from the population). More preferably the purity is about 70-75%, 75-80%, 80-85%; and most preferably the purity is about 85-90%, 90-95%, and 95-100%. Purity of the stromal cells, stem cells or their progenitors can be determined according to the genetic marker profile within a population. Therapeutic dosages can be readily adjusted by those skilled in the art (e.g., a decrease in purity may require an increase in dosage).

In other embodiments, mesenchymal stromal cells of the invention can be derived from suitable stem or progenitor cells. Stem cells of the present invention include mesenchymal stem cells. Mesenchymal stem cells, or “MSCs” are well known in the art. MSCs, originally derived from the embryonal mesoderm and isolated from adult bone marrow, can differentiate to form muscle, bone, cartilage, fat, marrow stroma, and tendon. During embryogenesis, the mesoderm develops into limb-bud mesoderm, tissue that generates bone, cartilage, fat, skeletal muscle and endothelium. Mesoderm also differentiates to visceral mesoderm, which can give rise to cardiac muscle, smooth muscle, or blood islands consisting of endothelium and hematopoietic progenitor cells. Primitive mesodermal or MSCs, therefore, could provide a source for a number of cell and tissue types. A number of MSCs have been isolated. (See, for example, Caplan, A., et al., U.S. Pat. No. 5,486,359; Young, H., et al., U.S. Pat. No. 5,827,735; Caplan, A., et al., U.S. Pat. No. 5,811,094; Bruder, S., et al., U.S. Pat. No. 5,736,396; Caplan, A., et al., U.S. Pat. No. 5,837,539; Masinovsky, B., U.S. Pat. No. 5,837,670; Pittenger, M., U.S. Pat. No. 5,827,740; Jaiswal, N., et al., (1997). J. Cell Biochem. 64(2):295-312; Cassiede P., et al., (1996). J Bone Miner Res. 9:1264-73; Johnstone, B., et al., (1998) Exp Cell Res. 1:265-72; Yoo, et al., (1998) J Bon Joint Surg Am. 12:1745-57; Gronthos, S., et al., (1994). Blood 84:4164-73); Pittenger, et al., (1999). Science 284:143-147.

Mesenchymal stem cells are believed to migrate out of the bone marrow, to associate with specific tissues. Enhancing the growth and maintenance of mesenchymal stem cells, in vitro or ex vivo will provide expanded populations that can be used to generate or regenerate tissues, including breast, skin, muscle, endothelium, bone, respiratory, urogenital, gastrointestinal connective or fibroblastic tissues.

Stem cells of the present invention also include embryonic stem cells. The embryonic stem (ES) cell has unlimited self-renewal and pluripotent differentiation potential (Thomson, J. et al. 1995; Thomson, J. A. et al. 1998; Shamblott, M. et al. 1998; Williams, R. L. et al. 1988; Orkin, S. 1998; Reubinoff, B. E., et al. 2000). These cells are derived from the inner cell mass (ICM) of the pre-implantation blastocyst (Thomson, J. et al. 1995; Thomson, J. A. et al. 1998; Martin, G. R. 1981), or can be derived from the primordial germ cells from a post-implantation embryo (embryonal germ cells or EG cells). ES and/or EG cells have been derived from multiple species, including mouse, rat, rabbit, sheep, goat, pig and more recently from human and human and non-human primates (U.S. Pat. Nos. 5,843,780 and 6,200,806).

Embryonic stem cells are well known in the art. For example, U.S. Pat. Nos. 6,200,806 and 5,843,780 refer to primate, including human, embryonic stem cells. U.S. Patent Applications Nos. 20010024825 and 20030008392 describe human embryonic stem cells. U.S. Patent Application No. 20030073234 describes a clonal human embryonic stem cell line. U.S. Pat. No. 6,090,625 and U.S. Patent Application No. 20030166272 describe an undifferentiated cell that is stated to be pluripotent. U.S. Patent Application No. 20020081724 describes what are stated to be embryonic stem cell derived cell cultures.

Stem cells of the present invention also include iPS cells. iPS cells are adult cells that have been genetically reprogrammed to an embryonic stem cell-like state by being forced to express genes and factors important for maintaining the defining properties of embryonic stem cells.

Isolated mesenchymal stromal cells as well as those derived from suitable stem or progenitor cells can be genetically altered to express desired nucleic acids according to methods known in the art, including all methods known to introduce transient and stable changes of the cellular genetic material. Genetic alteration of a mesenchymal stromal cell, stem or progenitor cell includes the addition of exogenous genetic material. Exogenous genetic material includes nucleic acids or oligonucleotides, either natural or synthetic, that are introduced into the cells.

Gene editing systems can be used to achieve genetic alteration of mesenchymal stromal cells, stem or progenitor cells. For example, the CRISPR/Cas system can be used to inactivate one or more nucleic acids, including CD248 and Cdh11 (Wiedenheft et al. (2012) Nature 482: 331-8). The CRISPR/Cas system has been modified for use in gene editing (silencing, enhancing or changing specific genes) in eukaryotes such as mice or primates. This is accomplished by, for example, introducing into the eukaryotic cell a plasmid containing a specifically designed CRISPR and one or more appropriate Cas. CRISPR/Cas systems for gene editing in eukaryotic cells typically involve (1) a guide RNA molecule (gRNA) comprising a targeting sequence (which is capable of hybridizing to the genomic DNA target sequence), and sequence which is capable of binding to a Cas, e.g., Cas9 enzyme, and (2) a Cas, e.g., Cas9, protein. The targeting sequence and the sequence which is capable of binding to a Cas, e.g., Cas9 enzyme, may be disposed on the same or different molecules. If disposed on different molecules, each includes a hybridization domain which allows the molecules to associate, e.g., through hybridization.

The CRISPR sequence, sometimes called a CRISPR locus, comprises alternating repeats and spacers. RNA from the CRISPR locus is constitutively expressed and processed into small RNAs. These comprise a spacer flanked by a repeat sequence. The RNAs guide other Cas proteins to silence exogenous genetic elements at the RNA or DNA level. Horvath et al. (2010) Science 327: 167-170; Makarova et al. (2006) Biology Direct 1: 7. The spacers thus serve as templates for RNA molecules, analogously to siRNAs. Pennisi (2013) Science 341: 833-836.

The CRISPR/Cas system can thus be used to modify, e.g., delete one or more nucleic acids, e.g., CD248 or a gene regulatory element of CD248, or introduce a premature stop which thus decreases expression of a functional CD248. The CRISPR/Cas system can alternatively be used like RNA interference, turning off the CD248 in a reversible fashion. In a mammalian cell, for example, the RNA can guide the Cas protein to a promoter of CD248 or Cdh11, sterically blocking RNA polymerases.

In another embodiment, the CRISPR/Cas system can be used to introduce one or more nucleic acids. The nucleic acid can be introduced into the cell along with the CRISPR/Cas system, e.g., DNA encoding Periostin and Pdgfra. This process can be used to integrate the DNA encoding Periostin and Pdgfra, e.g., as described herein, at or near the site targeted by the CRISPR/Cas system.

In other embodiments, the exogenous genetic material may also include a naturally occurring gene which has been placed under operable control of a promoter in an expression vector construct. Expression vectors include all those known in the art, such as cosmids, plasmids (e.g., naked or contained in liposomes), retrotransposons (e.g. piggyback, sleeping beauty), and viruses (e.g., lentiviruses, retroviruses, adenoviruses, and adeno-associated viruses) that can incorporate and deliver the recombinant polynucleotide.

Methods for producing viral expression vectors are known in the art. Typically, a disclosed virus is produced in a suitable host cell line using conventional techniques including culturing a transfected or infected host cell under suitable conditions so as to allow the production of infectious viral particles. Nucleic acids encoding viral genes and/or sequence(s) encoding, for example, periostin and pdgfra can be incorporated into plasmids and introduced into host cells through conventional transfection or transformation techniques. Exemplary suitable host cells for production of disclosed viruses include human cell lines such as HeLa, Hela-S3, HEK293, 911, A549, HER96, or PER-C6 cells. Specific production and purification conditions will vary depending upon the virus and the production system employed.

In some implementations, producer cells may be directly administered to a subject, however, in other implementations, following production, infectious viral particles are recovered from the culture and optionally purified. Typical purification steps may include plaque purification, centrifugation, e.g., cesium chloride gradient centrifugation, clarification, enzymatic treatment, e.g., benzonase or protease treatment, chromatographic steps, e.g., ion exchange chromatography or filtration steps.

In certain implementations, the expression vector is a viral vector. The term “virus” is used herein to refer any of the obligate intracellular parasites having no protein-synthesizing or energy-generating mechanism. Exemplary viral vectors include retroviral vectors (e.g., lentiviral vectors), adenoviral vectors, adeno-associated viral vectors, herpesviruses vectors, epstein-barr virus (EBV) vectors, polyomavirus vectors (e.g., simian vacuolating virus 40 (SV40) vectors), poxvirus vectors, and pseudotype virus vectors.

The virus may be a RNA virus (having a genome that is composed of RNA) or a DNA virus (having a genome composed of DNA). In certain implementations, the viral vector is a DNA virus vector. Exemplary DNA viruses include parvoviruses (e.g., adeno-associated viruses), adenoviruses, asfarviruses, herpesviruses (e.g., herpes simplex virus 1 and 2 (HSV-1 and HSV-2), epstein-barr virus (EBV), cytomegalovirus (CMV)), papillomoviruses (e.g., HPV), polyomaviruses (e.g., simian vacuolating virus 40 (SV40)), and poxviruses (e.g., vaccinia virus, cowpox virus, smallpox virus, fowlpox virus, sheeppox virus, myxoma virus). In certain implementations, the viral vector is a RNA virus vector. Exemplary RNA viruses include bunyaviruses (e.g., hantavirus), coronaviruses, ebolaviruses, flaviviruses (e.g., yellow fever virus, west nile virus, dengue virus), hepatitis viruses (e.g., hepatitis A virus, hepatitis C virus, hepatitis E virus), influenza viruses (e.g., influenza virus type A, influenza virus type B, influenza virus type C), measles virus, mumps virus, noroviruses (e.g., Norwalk virus), poliovirus, respiratory syncytial virus (RSV), retroviruses (e.g., human immunodeficiency virus-1 (HIV-1)) and toroviruses.

In certain implementations, the expression vector comprises a regulatory sequence or promoter operably linked to the nucleotide sequence encoding the exogenous sequence(s) encoding, for example, periostin and pdgfra. The term “operably linked” refers to a linkage of polynucleotide elements in a functional relationship. A nucleic acid sequence is “operably linked” when it is placed into a functional relationship with another nucleic acid sequence. For instance, a promoter or enhancer is operably linked to a gene if it affects the transcription of the gene. Operably linked nucleotide sequences are typically contiguous. However, as enhancers generally function when separated from the promoter by several kilobases and intronic sequences may be of variable lengths, some polynucleotide elements may be operably linked but not directly flanked and may even function in trans from a different allele or chromosome.

Additional exemplary promoters which may be employed include, but are not limited to, the retroviral LTR, the SV40 promoter, the human cytomegalovirus (CMV) promoter, the U6 promoter, or any other promoter (e.g., cellular promoters such as eukaryotic cellular promoters including, but not limited to, the histone, pol III, and β-actin promoters). Other viral promoters which may be employed include, but are not limited to, adenovirus promoters, TK promoters, and B19 parvovirus promoters. The selection of a suitable promoter will be apparent to those skilled in the art from the teachings contained herein.

In certain implementations, an expression vector is an adeno-associated virus (AAV) vector. AAV is a small, nonenveloped icosahedral virus of the genus Dependoparvovirus and family Parvovirus. AAV has a single-stranded linear DNA genome of approximately 4.7 kb. AAV is capable of infecting both dividing and quiescent cells of several tissue types, with different AAV serotypes exhibiting different tissue tropism. Numerous cell types are suitable for producing AAV vectors, including HEK293 cells, COS cells, HeLa cells, BHK cells, Vero cells, as well as insect cells (See e.g. U.S. Pat. Nos. 6,156,303, 5,387,484, 5,741,683, 5,691,176, 5,688,676, and 8,163,543, U.S. Patent Publication No. 20020081721, and PCT Publication Nos. WO00/47757, WO00/24916, and WO96/17947). AAV vectors are typically produced in these cell types by one plasmid containing the ITR-flanked expression cassette, and one or more additional plasmids providing the additional AAV and helper virus genes.

Non-limiting examples of AAV vectors include pAAV-MCS (Agilent Technologies), pAAVK-EF1α-MCS (System Bio Catalog #AAV502A-1), pAAVK-EF1α-MCS1-CMV-MCS2 (System Bio Catalog #AAV503A-1), pAAV-ZsGreen1 (Clontech Catalog #6231), pAAV-MCS2 (Addgene Plasmid #46954), AAV-Stuffer (Addgene Plasmid #106248), pAAVscCBPIGpluc (Addgene Plasmid #35645), AAVS1_Puro_PGK1_3×FLAG_Twin_Strep (Addgene Plasmid #68375), pAAV-RAM-d2TTA::TRE-MCS-WPRE-pA (Addgene Plasmid #63931), pAAV-UbC (Addgene Plasmid #62806), pAAVS1-P-MCS (Addgene Plasmid #80488), pAAV-Gateway (Addgene Plasmid #32671), pAAV-Puro_siKD (Addgene Plasmid #86695), pAAVS1-Nst-MCS (Addgene Plasmid #80487), pAAVS1-Nst-CAG-DEST (Addgene Plasmid #80489), pAAVS1-P-CAG-DEST (Addgene Plasmid #80490), pAAVf-EnhCB-lacZnls (Addgene Plasmid #35642), and pAAVS1-shRNA (Addgene Plasmid #82697). These vectors can be modified to be suitable for therapeutic use. For example, an exogenous nucleic acid sequence of interest can be inserted in a multiple cloning site, and a selection marker (e.g., puro or a gene encoding a fluorescent protein) can be deleted or replaced with another (same or different) exogenous gene of interest. Further examples of AAV vectors are disclosed in U.S. Pat. Nos. 5,871,982, 6,270,996, 7,238,526, 6,943,019, 6,953,690, 9,150,882, and 8,298,818, U.S. Patent Publication No. 2009/0087413, and PCT Publication Nos. WO2017075335A1, WO2017075338A2, and WO2017201258A1.

In certain implementations, the viral vector can be a retroviral vector. Examples of retroviral vectors include moloney murine leukemia virus vectors, spleen necrosis virus vectors, and vectors derived from retroviruses such as rous sarcoma virus, harvey sarcoma virus, avian leukosis virus, human immunodeficiency virus, myeloproliferative sarcoma virus, and mammary tumor virus. Retroviral vectors are useful as agents to mediate retroviral-mediated gene transfer into eukaryotic cells.

In certain implementations, the retroviral vector is a lentiviral vector. In certain implementations, the recombinant retroviral vector is a lentiviral vector including nucleic acids sequences encoding the two or more optimal epitopes. Exemplary lentiviral vectors include vectors derived from human immunodeficiency virus-1 (HIV-1), human immunodeficiency virus-2 (HIV-2), simian immunodeficiency virus (SIV), feline immunodeficiency virus (FIV), bovine immunodeficiency virus (BIV), Jembrana Disease Virus (JDV), equine infectious anemia virus (EIAV), and caprine arthritis encephalitis virus (CAEV).

Non-limiting examples of lentiviral vectors include pLVX-EF1alpha-AcGFP1-C1 (Clontech Catalog #631984), pLVX-EF1alpha-IRES-mCherry (Clontech Catalog #631987), pLVX-Puro (Clontech Catalog #632159), pLVX-IRES-Puro (Clontech Catalog #632186), pLenti6N5-DES™ (Thermo Fisher), pLenti6.2/V5-DES™ (Thermo Fisher), pLKO.1 (Plasmid #10878 at Addgene), pLKO.3G (Plasmid #14748 at Addgene), pSico (Plasmid #11578 at Addgene), pLJM1-EGFP (Plasmid #19319 at Addgene), FUGW (Plasmid #14883 at Addgene), pLVTHM (Plasmid #12247 at Addgene), pLVUT-tTR-KRAB (Plasmid #11651 at Addgene), pLL3.7 (Plasmid #11795 at Addgene), pLB (Plasmid #11619 at Addgene), pWPXL (Plasmid #12257 at Addgene), pWPI (Plasmid #12254 at Addgene), EF.CMV.RFP (Plasmid #17619 at Addgene), pLenti CMV Puro DEST (Plasmid #17452 at Addgene), pLenti-puro (Plasmid #39481 at Addgene), pULTRA (Plasmid #24129 at Addgene), pLX301 (Plasmid #25895 at Addgene), pHIV-EGFP (Plasmid #21373 at Addgene), pLV-mCherry (Plasmid #36084 at Addgene), pLionII (Plasmid #1730 at Addgene), pInducer10-mir-RUP-PheS (Plasmid #44011 at Addgene). These vectors can be modified to be suitable for therapeutic use. For example, a selection marker (e.g., puro, EGFP, or mCherry) can be deleted or replaced with a second exogenous nucleic acid sequence of interest. Further examples of lentiviral vectors are disclosed in U.S. Pat. Nos. 7,629,153, 7,198,950, 8,329,462, 6,863,884, 6,682,907, 7,745,179, 7,250,299, 5,994,136, 6,287,814, 6,013,516, 6,797,512, 6,544,771, 5,834,256, 6,958,226, 6,207,455, 6,531,123, and 6,352,694, and PCT Publication No. WO2017/091786.

In some implementations, the viral vector can be an adenoviral vector. Adenoviruses are medium-sized (90-100 nm), non-enveloped (naked), icosahedral viruses composed of a nucleocapsid and a double-stranded linear DNA genome. The term “adenovirus” refers to any virus in the genus Adenoviridiae including, but not limited to, human, bovine, ovine, equine, canine, porcine, murine, and simian adenovirus subgenera. Typically, an adenoviral vector is generated by introducing one or more mutations (e.g., a deletion, insertion, or substitution) into the adenoviral genome of the adenovirus so as to accommodate the insertion of a non-native nucleic acid sequence, for example, for gene transfer, into the adenovirus.

The adenoviral vector can be replication-competent, conditionally replication-competent, or replication-deficient. A replication-competent adenoviral vector can replicate in typical host cells, i.e., cells typically capable of being infected by an adenovirus. A conditionally-replicating adenoviral vector is an adenoviral vector that has been engineered to replicate under pre-determined conditions. For example, replication-essential gene functions, e.g., gene functions encoded by the adenoviral early regions, can be operably linked to an inducible, repressible, or tissue-specific transcription control sequence, e.g., a promoter. Conditionally-replicating adenoviral vectors are further described in U.S. Pat. No. 5,998,205. A replication-deficient adenoviral vector is an adenoviral vector that requires complementation of one or more gene functions or regions of the adenoviral genome that are required for replication, as a result of, for example, a deficiency in one or more replication-essential gene function or regions, such that the adenoviral vector does not replicate in typical host cells, especially those in a human to be infected by the adenoviral vector.

The replication-deficient adenoviral vector of the invention can be produced in complementing cell lines that provide gene functions not present in the replication-deficient adenoviral vector, but required for viral propagation, at appropriate levels in order to generate high titers of viral vector stock. Such complementing cell lines are known and include, but are not limited to, 293 cells (described in, e.g., Graham et al. (1977) J. Gen. Virol. 36: 59-72), PER.C6 cells (described in, e.g., PCT Publication No. WO1997/000326, and U.S. Pat. Nos. 5,994,128 and 6,033,908), and 293-ORF6 cells (described in, e.g., PCT Publication No. WO1995/034671 and Brough et al. (1997) J. Virol. 71: 9206-9213). Other suitable complementing cell lines to produce the replication-deficient adenoviral vector of the invention include complementing cells that have been generated to propagate adenoviral vectors encoding transgenes whose expression inhibits viral growth in host cells (see, e.g., U.S. Patent Publication No. 2008/0233650). Additional suitable complementing cells are described in, for example, U.S. Pat. Nos. 6,677,156 and 6,682,929, and PCT Publication No. WO2003/020879. Formulations for adenoviral vector-containing compositions are further described in, for example, U.S. Pat. Nos. 6,225,289, and 6,514,943, and PCT Publication No. WO2000/034444.

Additional exemplary adenoviral vectors, and/or methods for making or propagating adenoviral vectors are described in U.S. Pat. Nos. 5,559,099, 5,837,511, 5,846,782, 5,851,806, 5,994,106, 5,994,128, 5,965,541, 5,981,225, 6,040,174, 6,020,191, 6,083,716, 6,113,913, 6,303,362, 7,067,310, and 9,073,980.

Commercially available adenoviral vector systems include the ViraPower™ Adenoviral Expression System available from Thermo Fisher Scientific, the AdEasy™ adenoviral vector system available from Agilent Technologies, and the Adeno-X™ Expression System 3 available from Takara Bio USA, Inc.

In certain implementations, the viral vector can be a Herpes Simplex Virus plasmid vector. Herpes simplex virus type-1 (HSV-1) has been demonstrated as a potential useful gene delivery vector system for gene therapy. HSV-1 vectors have been used for transfer of genes to muscle, and have been used for murine brain tumor treatment. Helper virus dependent mini-viral vectors have been developed for easier operation and their capacity for larger insertion (up to 140 kb). Replication incompetent HSV amplicons have been constructed in the art. These HSV amplicons contain large deletions of the HSV genome to provide space for insertion of exogenous DNA. Typically, they comprise the HSV-1 packaging site, the HSV-1 “ori S” replication site and the IE 4/5 promoter sequence. These virions are dependent on a helper virus for propagation.

The methods of the invention can be used to treat any disease or disorder in which it is desirable to increase the amount of T cells. Frequently, subjects in need of the inventive treatment methods will be those undergoing or expecting to undergo an immune cell depleting treatment such as chemotherapy. Most chemotherapy agents act by killing all cells going through cell division. Thus, methods of the invention can be used, for example, to treat patients requiring a bone marrow transplant or a hematopoietic stem cell transplant, such as cancer patients undergoing chemo and/or radiation therapy. Methods of the present invention are particularly useful in the treatment of patients undergoing chemotherapy or radiation therapy for cancer, including patients suffering from myeloma, non-Hodgkin's lymphoma, Hodgkins lymphoma, or leukaemia.

Disorders treated by methods of the invention can be the result of an undesired side effect or complication of another primary treatment, such as radiation therapy, chemotherapy, or treatment with an immune suppressive drug, such as zidovadine, chloramphenical or gangciclovir. Such disorders include neutropenias, anemias, thrombocytopenia, and immune dysfunction.

A reduced level of immune function compared to a normal subject can result from a variety of disorders, diseases infections or conditions, including immunosuppressed conditions due to leukemia, renal failure; autoimmune disorders, including, but not limited to, systemic lupus erythematosus, rheumatoid arthritis, auto-immune thyroiditis, scleroderma, inflammatory bowel disease; various cancers and tumors; viral infections, including, but not limited to, human immunodeficiency virus (HIV); bacterial infections; and parasitic infections and may occur as a consequence of aging.

Accordingly, the present invention provides methods of treating disease and/or disorders or symptoms thereof which comprise administering a therapeutically effective amount of a composition comprising Periostin+Pdgfra+ mesenchymal stromal cells described herein to a subject (e.g., a mammal, such as a human). Thus, one embodiment is a method of treating a subject having a disease characterized by a lack of T-cells or by an altered complexity of T cell receptors within a population of T cells. The method includes the step of administering to the subject a therapeutic amount of Periostin+Pdgfra+ mesenchymal stromal cells or mesenchymal stem cells expressing CCL19 or a mixture comprising such cell types, or CCL19 itself sufficient to treat a disease or disorder or symptom thereof, under conditions such that the disease or disorder is treated. Identifying a subject in need of such treatment can be in the judgment of a subject or a health care professional and can be subjective (e.g. opinion) or objective (e.g. measurable by a test or diagnostic method).

Periostin+Pdgfra+ mesenchymal stromal cells are administered according to methods known in the art. Such compositions may be administered by any conventional route, including injection or by gradual infusion over time. The administration may, depending on the composition being administered, for example, be, intrathymic, pulmonary, intravenous, intraperitoneal, intramuscular, intracavity, subcutaneous, or transdermal. The Periostin+Pdgfra+ mesenchymal stromal cells are administered in “effective amounts”, or the amounts that either alone or together with further doses produces the desired therapeutic response. Administered cells of the invention can be autologous (“self”) or non-autologous (“non-self,” e.g., allogeneic, syngeneic or xenogeneic). Generally, administration of the cells can occur within a short period of time following treatment (e.g. 1, 2, 5, 10, 24 or 48 hours after treatment) and according to the requirements of each desired treatment regimen. For example, where radiation or chemotherapy is conducted prior to administration, treatment, and transplantation of cells of the invention should optimally be provided within about one month of the cessation of therapy. However, transplantation at later points after treatment has ceased can be done with derivable clinical outcomes.

Periostin+Pdgfra+ mesenchymal stromal cells can be combined with pharmaceutical excipients known in the art to enhance preservation and maintenance of the cells prior to administration. In some embodiments, cell compositions of the invention can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the cells utilized in practicing the present invention in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like.

The compositions can be isotonic, i.e., they can have the same osmotic pressure as blood and lacrimal fluid. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

A method to potentially increase cell survival when introducing the cells into a subject in need thereof is to incorporate cells of interest into a biopolymer or synthetic polymer. Depending on the subject's condition, the site of injection might prove inhospitable for cell seeding and growth because of scarring or other impediments. Examples of biopolymer include, but are not limited to, cells mixed with fibronectin, fibrin, fibrinogen, thrombin, collagen, and proteoglycans. This could be constructed with or without included expansion or differentiation factors. Additionally, these could be in suspension, but residence time at sites subjected to flow would be nominal. Another alternative is a three-dimensional gel with cells entrapped within the interstices of the cell biopolymer admixture. Again, expansion or differentiation factors could be included with the cells. These could be deployed by injection via various routes described herein.

Those skilled in the art will recognize that the components of the compositions should be selected to be chemically inert and will not affect the viability or efficacy of the stem cells or their progenitors as described in the present invention. This will present no problem to those skilled in chemical and pharmaceutical principles, or problems can be readily avoided by reference to standard texts or by simple experiments (not involving undue experimentation), from this disclosure and the documents cited herein.

One consideration concerning the therapeutic use of cells is the quantity of cells necessary to achieve an optimal effect. Different scenarios may require optimization of the amount of cells injected into a tissue of interest. Thus, the quantity of cells to be administered will vary for the subject being treated. The precise determination of what would be considered an effective dose may be based on factors individual to each patient, including their size, age, sex, weight, and condition of the particular patient. As few as 100-1000 cells can be administered for certain desired applications among selected patients. Therefore, dosages can be readily ascertained by those skilled in the art from this disclosure and the knowledge in the art.

The skilled artisan can readily determine the amount of cells and optional additives, vehicles, and/or carrier in compositions and to be administered in methods of the invention. Of course, for any composition to be administered to an animal or human, and for any particular method of administration, it is preferred to determine therefore: toxicity, such as by determining the lethal dose (LD) and LD₅₀ in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

The present invention also provides methods of treating disease and/or disorders or symptoms thereof which comprise administering a therapeutically effective amount of a composition comprising Ccl19 (C-C motif chemokine ligand 19) into a T-cell producing tissue or fluid of the subject, such as the thymus. Ccl19 is a cytokine that plays a role in normal lymphocyte recirculation and homing. It also plays an important role in trafficking of T cells in thymus, and in T cell and B cell migration to secondary lymphoid organs. It is expressed in the Periostin+Pdgfra+ mesenchymal stromal cells of the invention.

Ccl19 can be administered in effective amounts through any suitable mode of administration known in the art (e.g., injection or infusion). The effective amount will depend upon the mode of administration, the particular condition being treated and the desired outcome. It may also depend upon the stage of the condition, the age and physical condition of the subject, the nature of concurrent therapy, if any, and like factors well known to the medical practitioner. For therapeutic applications, it is that amount sufficient to achieve a medically desirable result (an increase in T cell production). Generally, doses of active Cc119 polypeptide compounds of the present invention would be from about 0.01 mg/kg per day to about 1000 mg/kg per day. It is expected that doses ranging from about 50 to about 2000 mg/kg will be suitable. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of the Ccl19 compositions of the present invention.

The present invention is additionally described by way of the following illustrative, non-limiting Examples that provide a better understanding of the present invention and of its many advantages.

### Examples

The following Examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following Examples do not in any way limit the invention.

The Materials and Methods used to conduct the assays in the following Examples are described in detail herein below.

Animals: Male and female C57Bl/6 mice 8 weeks of age were used for all transplantation and sequencing experiments. B6.SJL-Ptprca Pepcb/BoyJ (CD45.1) and C57BL/6-Tg(UBC-GFP)30Scha/J mice were used as donors for bone marrow transplantations. B6; 129S-Penktm2(cre)Hze/J mice were crossed with B6.Cg-Gt(ROSA)26Sortm14(CAG-tdTomato)Hze/J to generate donors for mesenchymal stromal cell (MSC) transfers. All mice were obtained from Jackson Laboratories and all animal experimentation was carried out in accordance with national and institutional guidelines.

Tissue collection and processing: All human tissue specimens were collected with institutional review board (IRB) approval. The tissue was processed immediately upon isolation to ensure highest possible cell quality. Murine samples were cut into fine pieces and digested in Medium 199 (M199, Gibco) with 2% (v/v) fetal bovine serum (FBS, Gibco), Liberase (0.5WU/ml, Roche) and DNAse I (0.1 KU, Invitrogen) 3×15 minutes at 37° C. under constant agitation. Human samples were processed by digestion with M199 with 2% FBS, DNAse I (0.1 KU) and 2 mg/ml Stemxyme 1 (Worthington) for 2×30 minutes at 37° C. under constant agitation. For the last 30 minutes the samples were digested with the Stemxyme/DNAse I cocktail in combination with 0.125% Trypsin (Gibco). All samples were digested in the presence of RNase inhibitors (RNasin (Promega) and RNase OUT (Invitrogen).

FACS sorting for single-cell RNA sequencing: After blocking with anti-human CD16/32 Fc-block (BD Biosciences) for 10 minutes at 4° C., human single cell suspensions were stained with Lineage cocktail-FITC, CD66b-FITC, CD45-BV711, CD235a-BV711, CD8a-APC/Cy7 and CD4-BV605 (all from BD Biosciences). Mouse samples were also blocked with anti-mouse CD16/32 Fc-block (BD Biosciences) for 10 minutes at 4° C., followed by staining with CD45-PE/Cy7 and Ter119-PE (Both from BioLegend). Samples were stained for 45 minutes at 4° C. under constant agitation. For detection of dead cells 7-AAD (ThermoFisher) was added to the samples immediately before analysis. Flow sorting for live and non-hematopoietic cells (7-AAD, CD45-CD235a/Terl 19-Lineage-) was performed on a BD FACS Aria III equipped with a 70 um nozzle (BD Biosciences).

FACS sorting and analysis of thymus stromal cell populations: For analysis of various thymus stromal cell populations human samples were stained with Lineage cocktail-FITC, CD66b-FITC, CD45-BV711, CD235a-BV711, CD8a-APC/Cy7 and CD4-BV605 in combination with CD326-BV421 (BD Bioscience) and CD31-PE/Dazzle594 (BioLegend). Murine stromal cell types were characterized and sorted by surface staining for CD45-APC/Cy7 and Ter119-APC/Cy7 (both from BD Biosciences) as well as CD31-BUV737, CD326-BV77, and CD140a-BV785 (all from BD Biosciences). Itgb5, CD9912 and CD248 (R&D Systems) were conjugated in house to PE/Cy7 and APC (Abcam) respectively and also used for some of the stromal cell sorts.

Single-cell RNA sequencing: Sorted thymus stromal cells were encapsulated into emulsion droplets using the Chromium Controller (10× Genomics). scRNA sequencing libraries were subsequently prepared using Chromium Single Cell 3′ v2 Reagent kit (10× Genomics). Libraries were diluted to 4 nM and pooled before sequencing on the NextSeq 500 Sequencing system (Illumina).

Transplantation of bone marrow, lymphoid progenitors and MSCs: 8 weeks old C57Bl/6 mice received a single dose of 9.5 Grey 12-24 hours prior to the transplantation. For lymphoid progenitor transplantations bone marrow from C57BL/6-Tg(UBC-GFP)30Scha/J donors was lineage depleted (Miltenyi) following the manufacturer's instructions. The cells were subsequently stained with biotinylated lineage antibodies (CD3e, B220, CD4, CD8a, Gr-1, Cd11b), cKit-APC and CD135-BV421 for 30 minutes at 4° C. This was followed by a 15 minute incubation with Streptavidin-PE/Cy7. Lineage—CD135+ cKit+GFP+ lymphoid progenitors were sorted on a BD FACS Aria III and 40 000 cells were injected into each lethally irradiated recipient along with 10⁶ nucleated whole bone marrow cells from B6.SJL-Ptprca Pepcb/BoyJ donors. In the case of the adoptive transfer of MSCs recipients were irradiated 12 hours prior to the transfer to ensure that the thymus would be of a size that enables intrathymic injections. 2000-10 000 MSCs (CD45-Ter119-CD31-CD326-CD248+CD9912+Itgb5+CD140+) were injected intrathymically along with a retro-orbital injection of 10⁶ nucleated whole bone marrow cells from B6.SJL-Ptprca Pepcb/BoyJ mice.

Tissue clearing and 2-photon imaging: For imaging of native fluorescence the tissue was fixed in vivo by infusion of 4% paraformaldehyde (PFA, Electron Microscopy Sciences) followed by an additional 6 hour incubation with 4% PFA. The tissue was dehydrated through consecutive incubation steps in increasing concentration of tert-butanol solutions (Sigma, v/v, 50%, 70%, 80%, 90% and 100%). Lipids were removed by a 45-minute exposure to dichloremethane (Sigma). Lastly refractive index matching was achieved my incubation in benzyl alcohol, benzyl benzoate and diphenyl ether (BABB-D4, Sigma, 26%:53%:20%). Before imaging the sample is mounted between 2 coverslips, submerged in BABB-D4. Images were acquired on a Olympus FVMPE-RS multiphoton imaging platform (Olympus).

### Example 1. Single-Cell Sequencing of Human and Mouse Thymus Identifies Mesenchymal Cell Subsets with Distinct T Cell Supportive Signatures

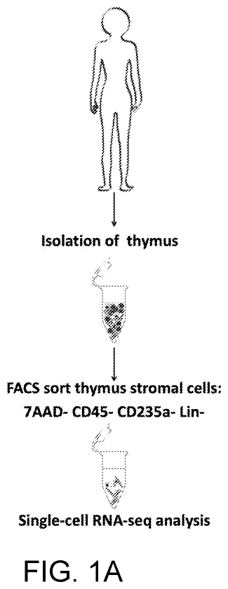

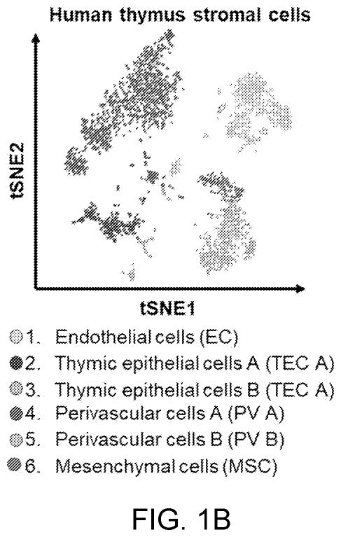

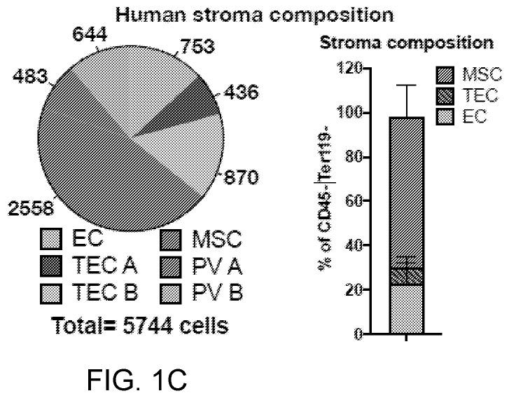

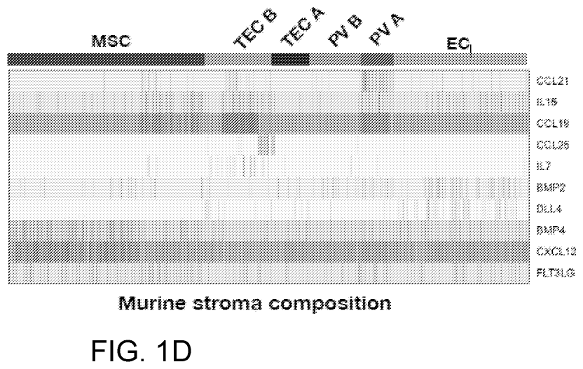

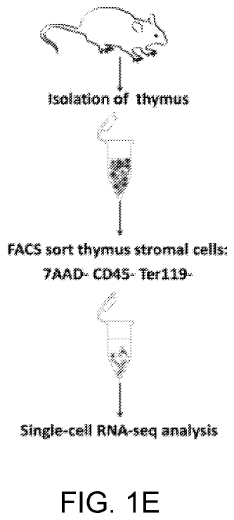

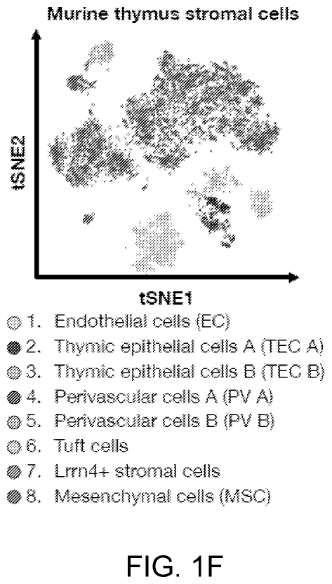

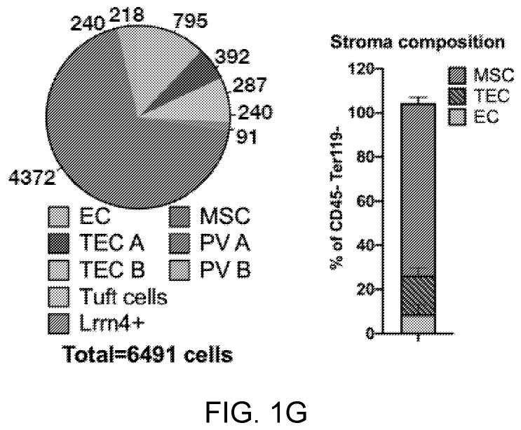

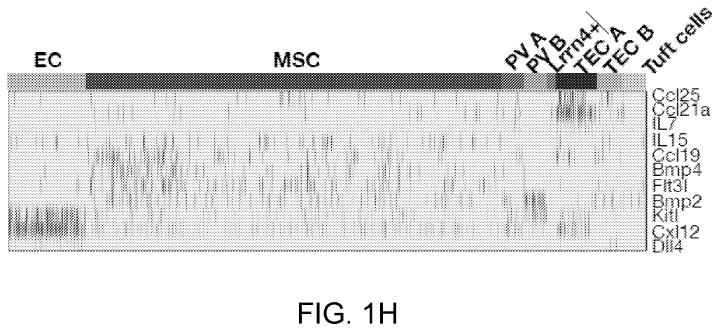

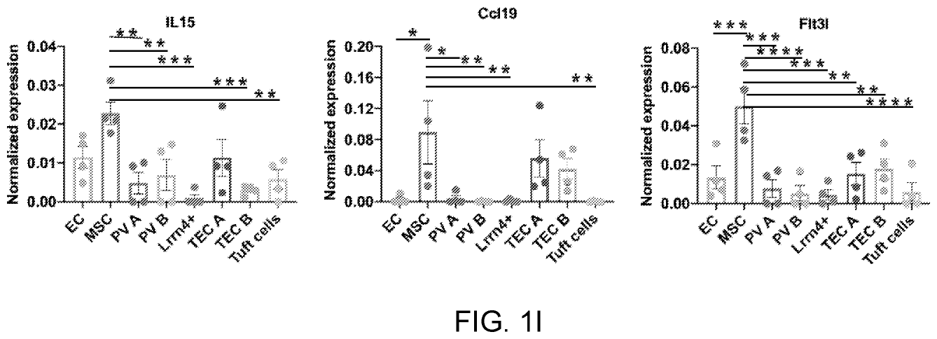

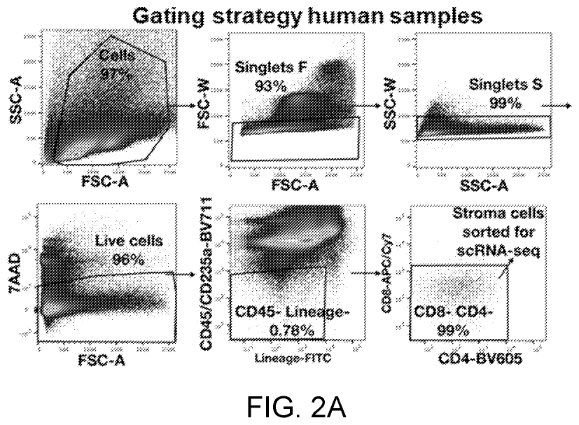

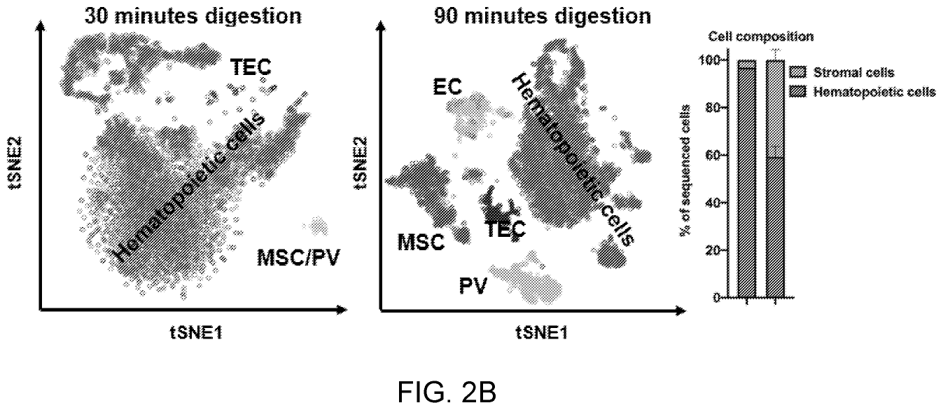

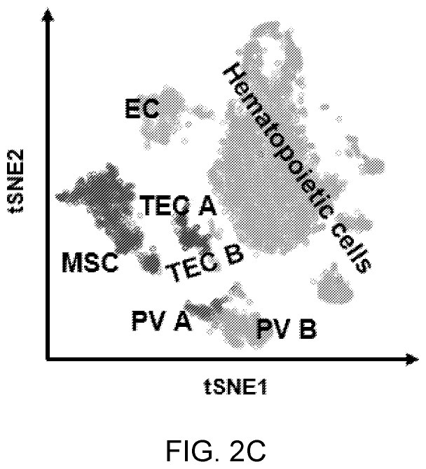

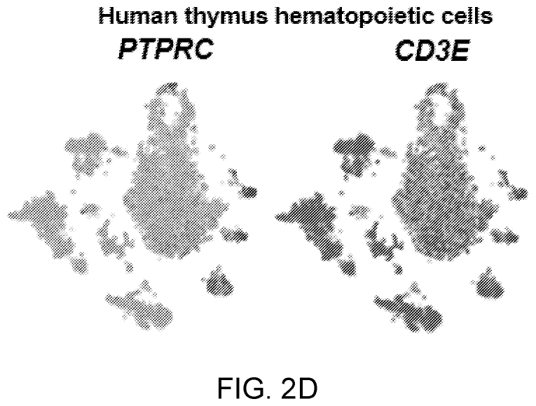

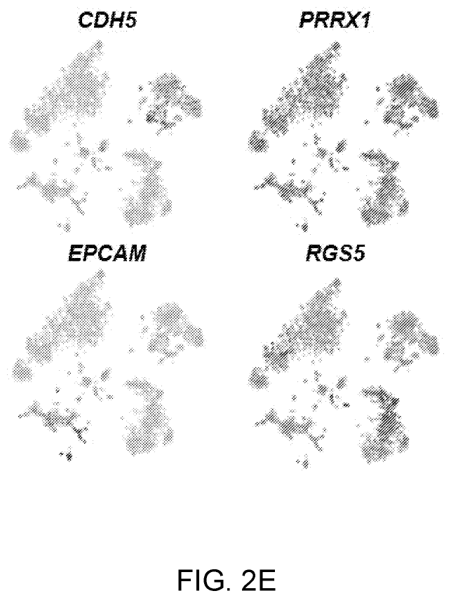

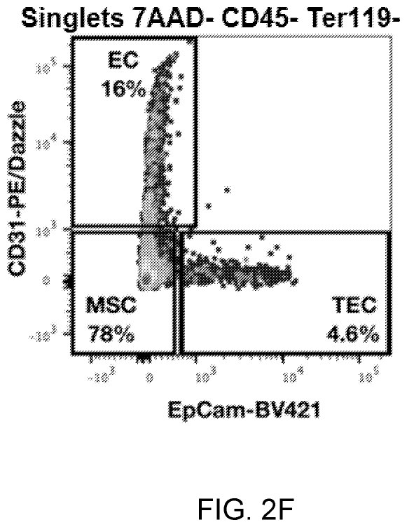

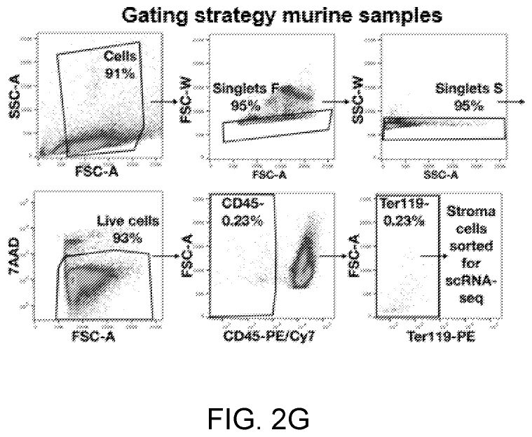

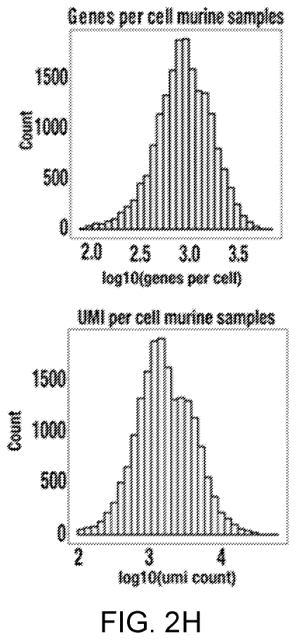

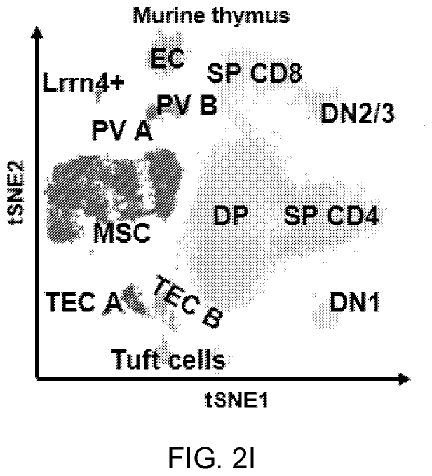

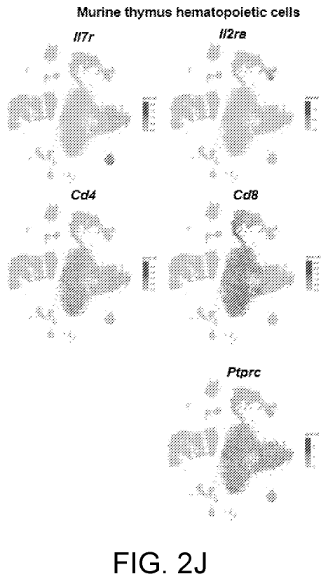

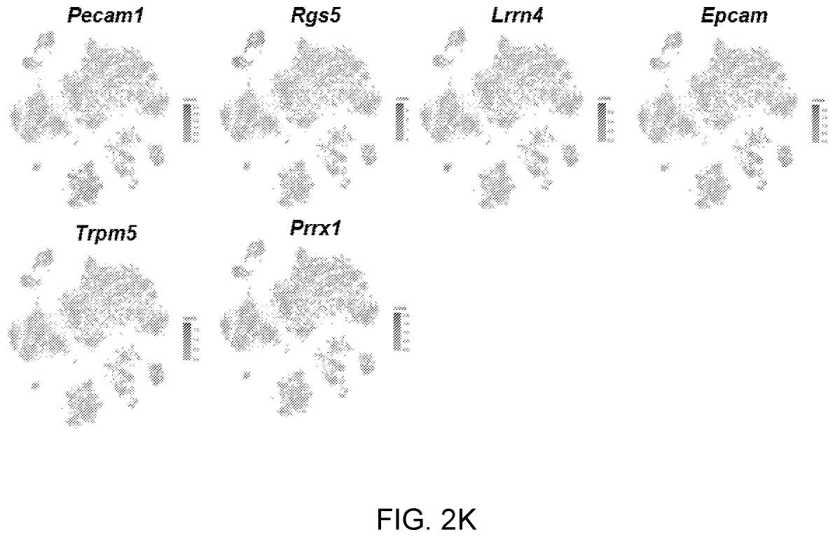

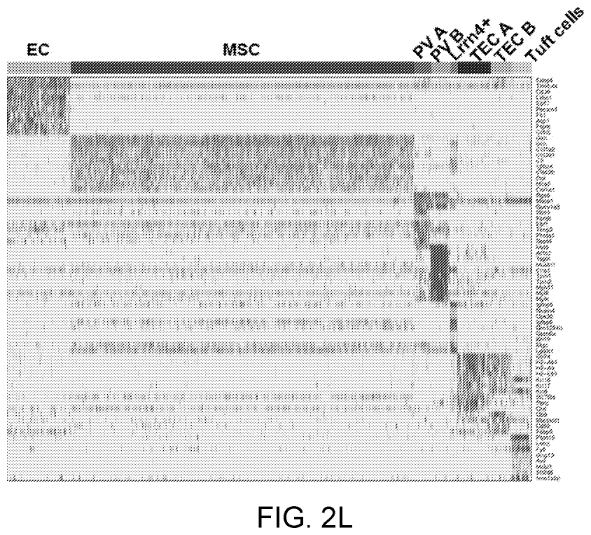

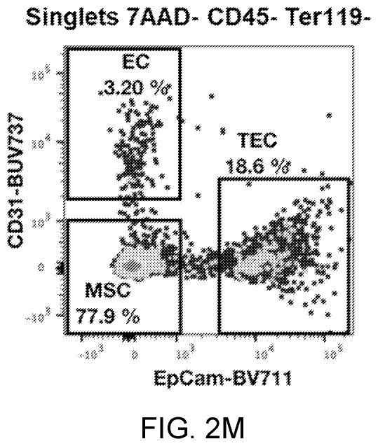

Inefficient T cell reconstitution following a bone marrow transplantation is a major cause of morbidity and mortality. Successful reestablishment of T cell mediated immunity is in turn entirely dependent on the regenerative ability of the thymus. Yet the mechanisms underlying impaired thymic recovery are poorly defined. In particular, regeneration of the stromal cells that support T cell development remain incompletely understood. In order to characterize the thymic microenvironment CD45-CD235-CD45-Lin-thymic stromal cells were isolated and performed single-cell RNA sequencing on 1 human thymus samples (FIG. 1A, FIG. 2A)). Initial efforts demonstrated the importance of digestion conditions for successful isolation of thymus stromal cells from human tissue. A shorter digestion yielded poor stromal cell enrichment and low cell type diversity as compared to a more extended protocol (FIG. 2B). Flow sorting of non-hematopoietic cells always results in contamination of blood cells (FIGS. 2C and D), all cells expressing PTPRC and CD3E were therefore removed from further analysis (FIG. 2D).

In the stromal cell compartment, six cell populations were subsequently identified with distinct expression patterns: endothelial cells (CDH5), mesenchymal stromal cells (PRRX1), two types of thymic epithelial cells (EPCAM), and two types of perivascular cells (RGS5). (FIG. 1B, FIG. 2E). The proportions of different populations were similar between samples, an observation that was largely confirmed by flow cytometry (FIG. 1C and FIG. 2F). Interestingly, the largest fraction of stromal cells were found to be made up of the PRRXJ expressing mesenchymal stromal cells (MSCs) (FIG. 1C), a population of cells that, despite their abundance, has received little attention in the context of T cell development in the thymus.

The main function of thymic stromal cells is to provide factors that recruit, sustain and commit hematopoietic progenitors to the T cell lineage. Many of the molecules that partake in this process have been defined. Assessment of which cell types express these lymphopoietic factors, revealed some expected pairings. Thymic epithelium (TEC) were found to be particularly enriched in the T cell progenitor recruiting chemokines CCL21 and CCL25 (FIG. 1D). Notably however, human thymic MSCs appeared to express high levels of several well-established regulators of lymphoid cell development, including FLT3LG, CCL19, and IL15 (FIG. 1D). Suggesting that the substantial pool of thymus mesenchymal cells may be important contributors to T cell development.

To further understand and characterize the identified populations in the humans, scRNA-seq was performed on resting state thymus of 8 weeks old mice (FIG. 1E and FIG. 2G). A total of 4 samples were sequenced that after quality control and filtering of hematopoietic cells yielded a total of 6491 murine stromal cells (FIGS. 2H, 1I and 1J). The thymus stromal cell populations found in human were all present in the mouse as well: endothelial cells (Pecam1), mesenchymal stromal cells (Prrx1), two types of perivascular (Rgs5) and thymic epithelial (Epcam) cells, respectively (FIG. 1F, FIGS. 2K and 2L). In addition, the murine thymus contains two other stromal subsets. The recently described thymic Tuft cells defined by expression of Trpm5 as well as IL25 (FIG. 1F, FIGS. 2K and 2L). A small population of cells were also found to express Lrrn4, a marker previously associated with mesothelial stem- and progenitors (FIG. 1F, F2K and 2L). These discrepancies in thymus stromal cell content could reflect an actual interspecies difference but may well be due to inherent differences in sample preparation and sample source. Most human samples were for instance from infants whereas the murine tissue was isolated from adults. Nevertheless, studies were continued using adult mice, as this a more relevant population in which to study thymic regeneration.

Just as was seen in human samples, the largest fraction of stromal cells in mice were found to be MSCs, determined by scRNA sequencing as well as flow cytometric analysis (FIG. 1G, FIG. 2M). Key thymocyte supportive factors were also found to be enriched in murine, thymic MSCs (F1H). In fact, IL-15, Flt31, Cc119 and Bmp4 were expressed at significantly higher levels in the MSC subset compared to all other stromal cell types (FIG. 1I). Thus, T cell supportive MSCs appear to be present in human as well as murine thymic tissue.

### Example 2. Periostin+ Thymic MSCs Preferentially Express T Cell Regulators

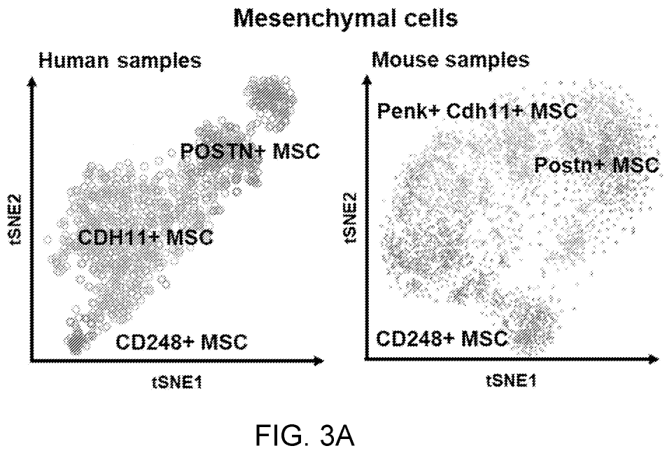

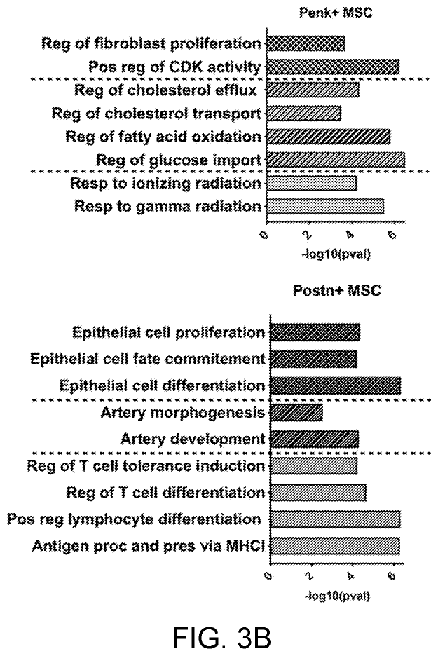

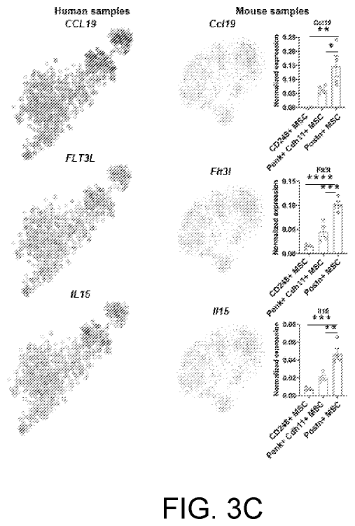

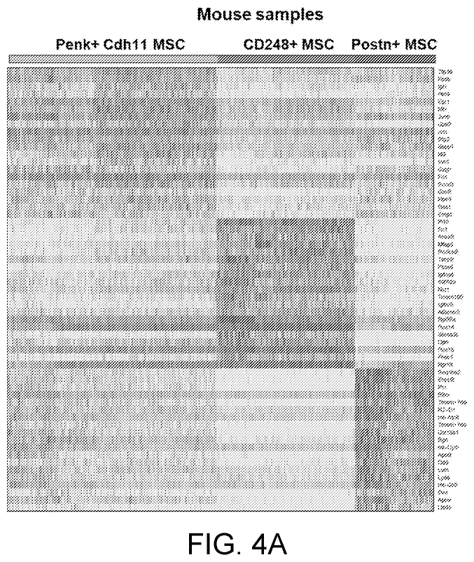

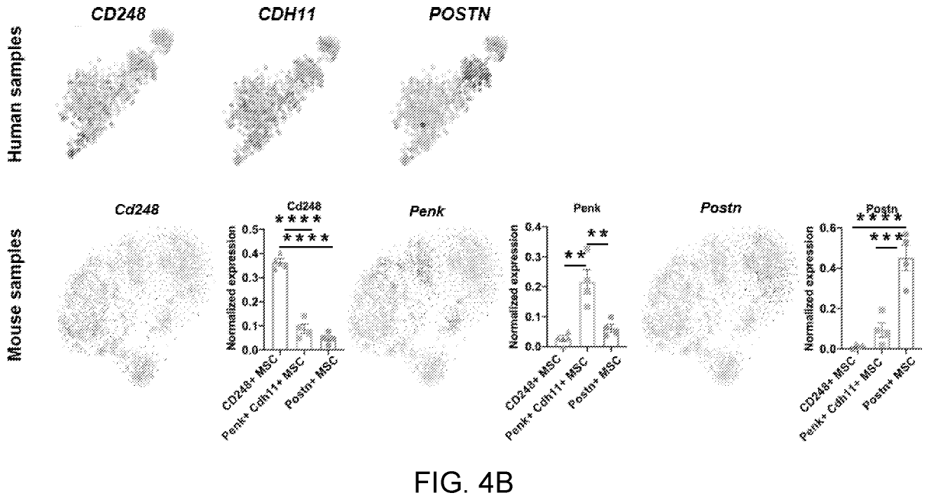

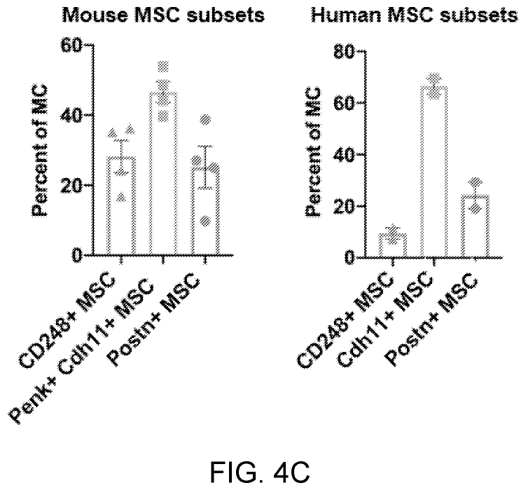

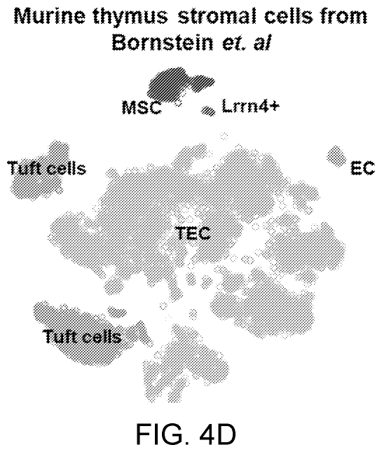

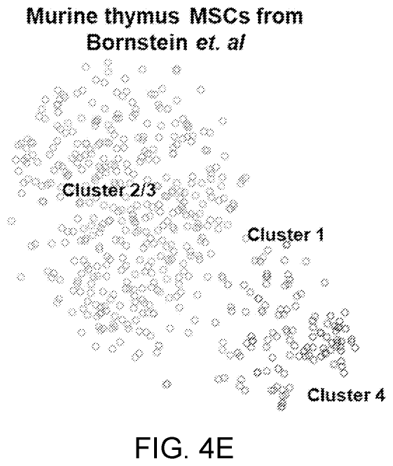

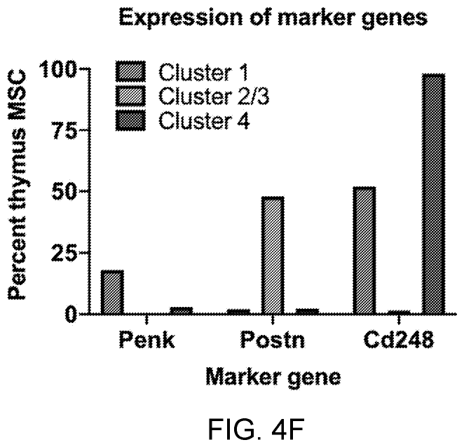

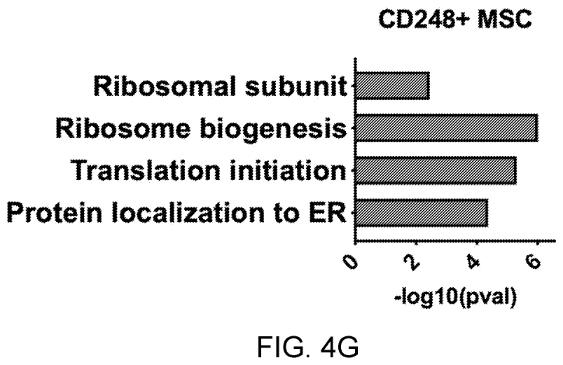

The MSC compartment was further explored, identifying three distinct subpopulations in both human and murine thymus (FIG. 3A, FIG. 4A). Both species were found to have a CD248+ and Postn+ MSC population, albeit at varying frequencies (FIG. 3A, FIGS. 4B and 4C). The third MSC subset was found to be characterized by CDH11 expression in human whereas in murine samples the cells defined by Cdh11 and Penk (FIG. 3A, FIGS. 4B and 4C). Comparison with a previously published data set of murine thymus stroma further validated the existence of three MSC subpopulations (FIGS. 4D and 4E). The relative abundance of MSCs overall as well as the three subtypes was found to be different (FIGS. 4D and 4E). However, as thymic epithelial cells were the primary focus of that study, an alternative isolation protocol was used, likely explaining the differences. Notably, Cd248, Penk and Posn expressing MSCs were also found in this data set (FIG. 4F).

GO term analysis of the murine samples further revealed potentially distinct functions among the MC subtypes. CD248+ MSCs were found to primarily be enriched for terms involving protein translation and secretion (FIG. 4G). This, in combination with the elevated expression of multiple extracellular matrix components (Fn1 and Ogn) displayed by these cells (FIG. 4A), is suggestive of a fibroblastic function for these cells. Penk+ Cdh11+ MCs were on the other hand found to be characterized by terms associated with adipogenesis and stress responses (FIG. 3B). This may be of particular interest as the epithelial compartment in the aging thymus is gradually being replaced by adipocytes through an unknown process. The expression of epithelial regulatory programs in Postn+ MSCs (FIG. 3B) is in line with what has previously been known about the function of thymic MSC, where mesenchymal lineage cells during embryogenesis partake in the recruitment of epithelial progenitors. Postn+ cells also displayed significant activation of angiogenesis pathways (FIG. 3B), suggesting that these cells may play a key role in regulating other thymus stromal cell types. Most importantly though, Postn+ MSCs were found to be the subtype significantly enriched in T cell development and differentiation terms (FIG. 3B). This observation was further confirmed by the fact that both human and murine Postn+ MSCs expressed lymphopoietic cytokines Ccl19, Flt31 and IL15 at significantly higher levels than the other MSC subpopulations (FIG. 3C). Indicating that Postn+ MSCs are responsible for the majority of interactions with developing T cells in the thymus.

### Example 3. Loss of Periostin+ MSCs Following Radiation Conditioning

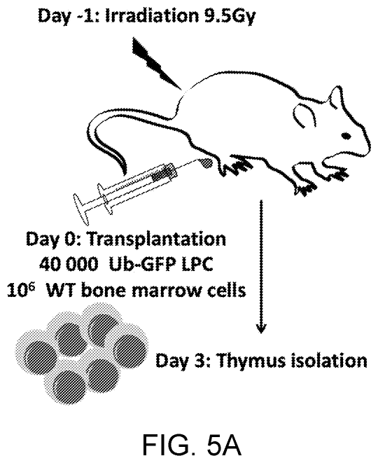

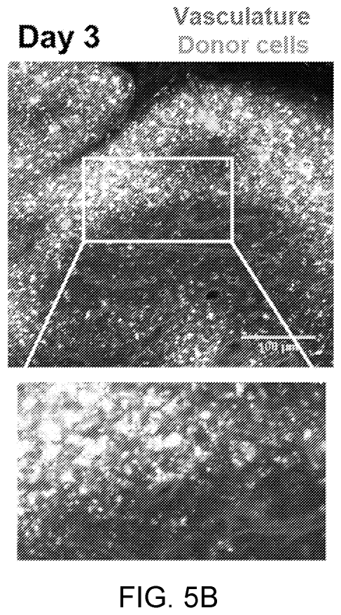

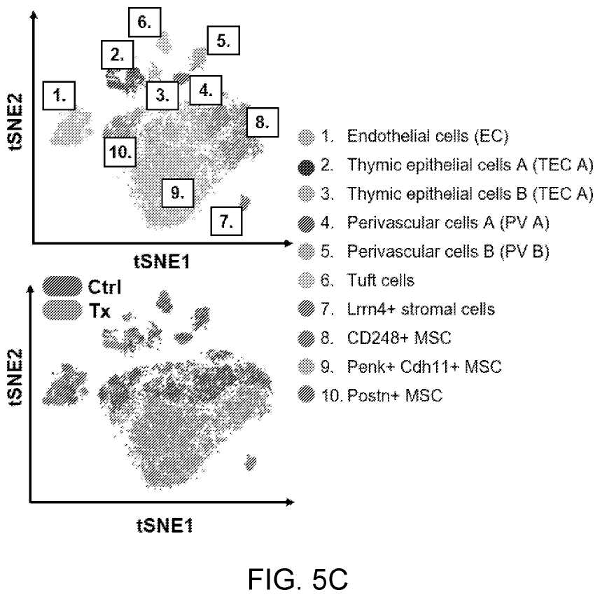

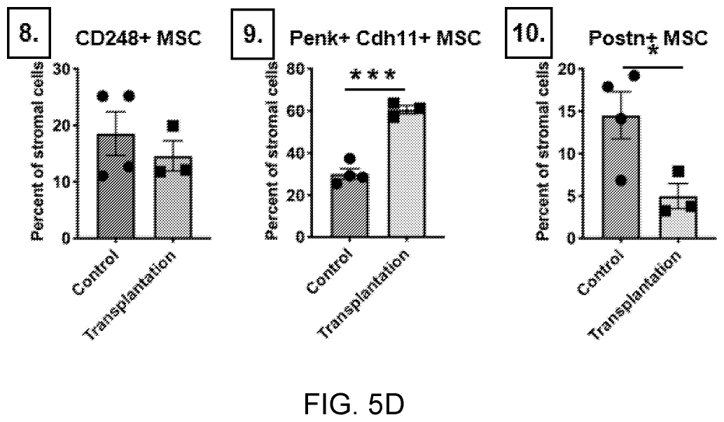

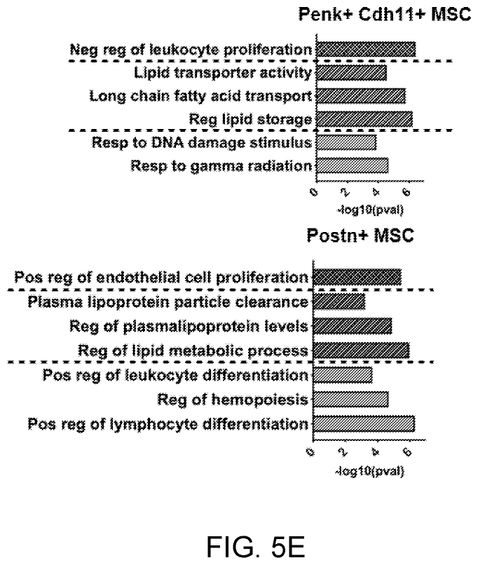

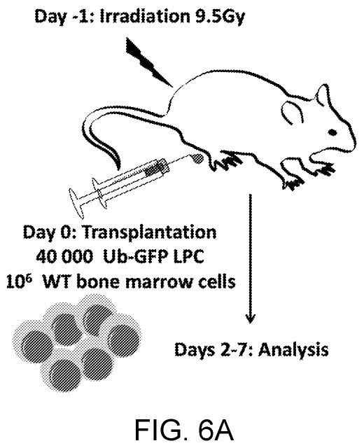

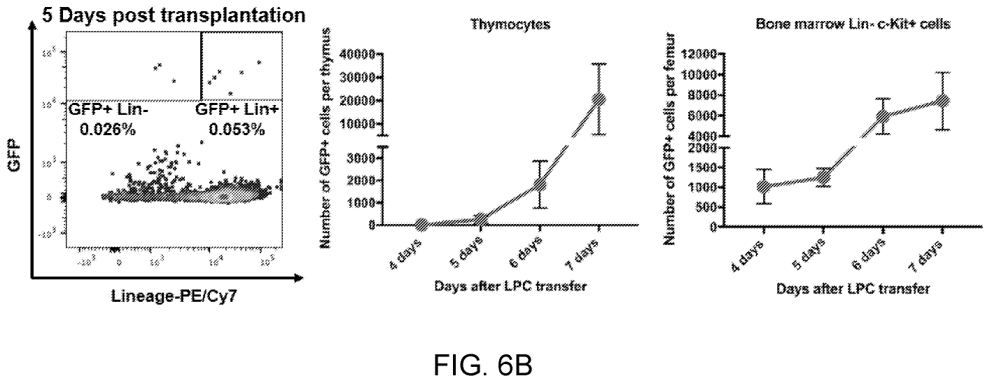

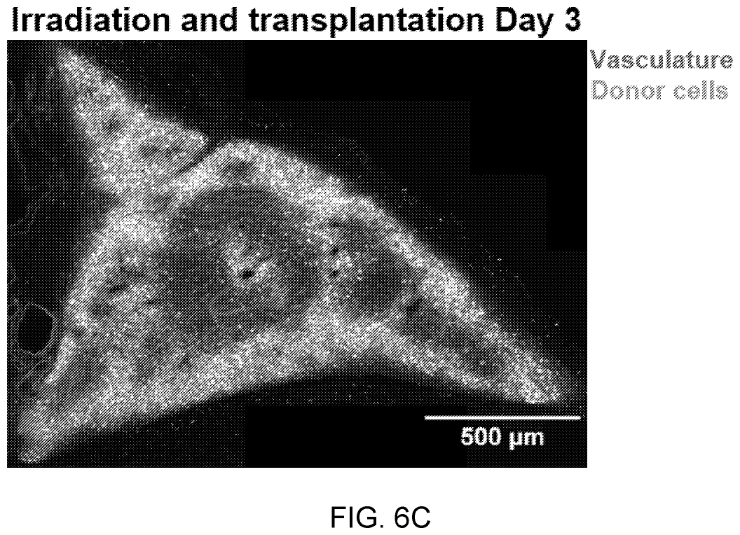

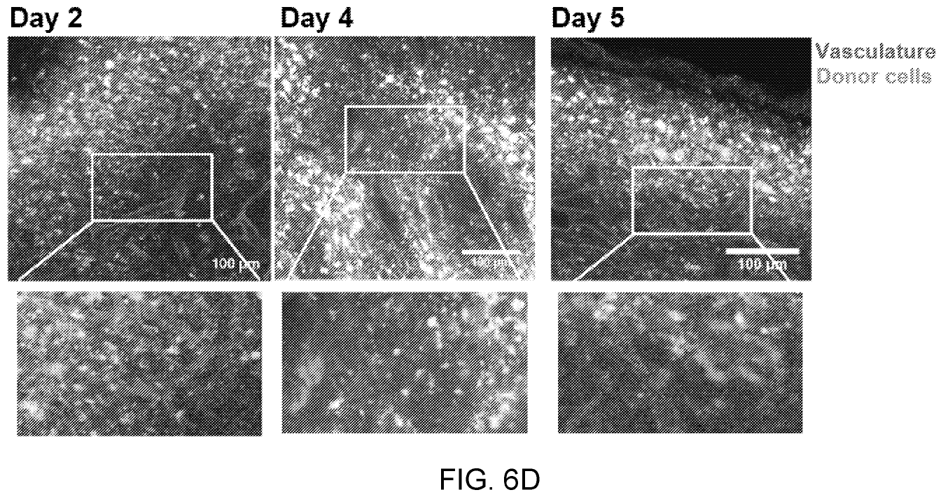

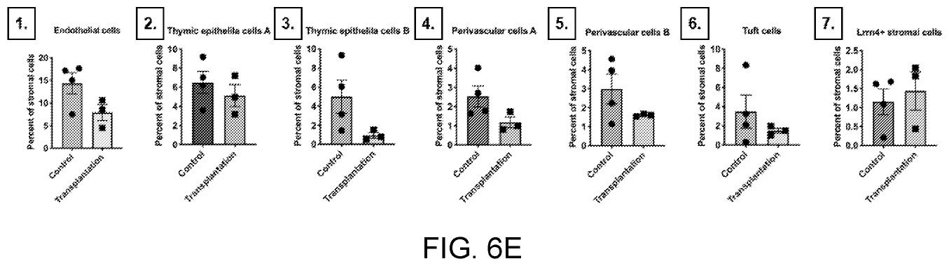

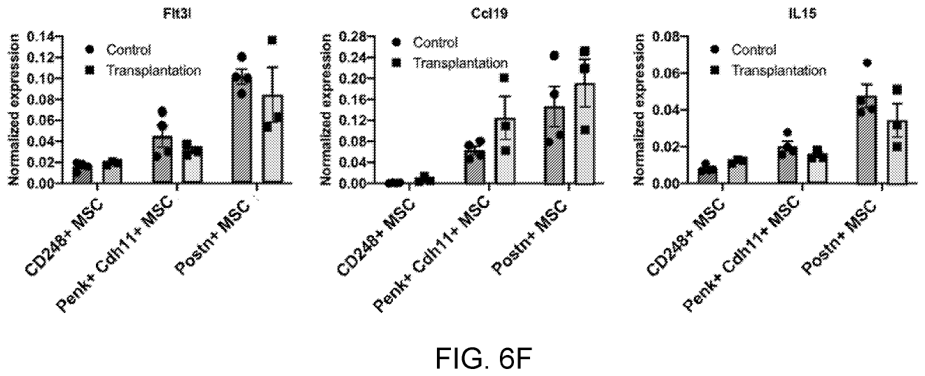

As thymus regeneration is of particular interest in the context of bone marrow transplantation, we wanted to compare our steady state scRNA sequencing with samples that had undergone cytotoxic conditioning and transplantation. A major hurdle in early thymic regeneration is inefficient recruitment of T cell progenitors from the bone marrow. In order to better understand what is missing in the microenvironment at this stage, we aimed to sample the thymus stroma at the timepoint when T cell progenitors first seed the tissue after the transplantation. To this end we transplanted 40 000 GFP labeled lymphoid progenitor cells (LPC, lineage-cKit+CD135+) into lethally irradiated recipient mice along with 1 million helper marrow cells and attempted to track thymic seeding using flow cytometry (FIGS. 6A and B). This turned out to be an unreliable approach. Although GFP+ cells were readily found in the marrow, few, if any, could be detected in the thymus at early timepoints after transplantation (FIG. 6B). Additionally, many of the cells were positive for lineage defining markers (FIG. 6B), suggesting they were not early thymic progenitors (ETPs). Consequently, tracing was switched to recent thymic settlers by tissue clearing, as this enables imaging from top to bottom with minimal loss of material (FIG. 5A, FIG. 6C). This revealed that rare, GFP+ cells were first detected in the thymus 3 days following the transplantation (FIG. 5B, FIG. 6D), whereas the tissue was found to contain an abundance of immigrated cells at later stages (FIG. 6D). Thus, it appears as though the thymus seeding is initiated 3 days post-transplantation and this was selected as the timepoint for our scRNA sequencing analysis of thymus stromal cells.

As was done for the steady state analysis, CD45− Ter119− cells were sorted from 8 weeks old mice that received a single, lethal dose of irradiation 4 days prior, and a bone marrow graft of 40 000 GFP+ LPCs and unlabeled helper marrow, 3 days before the isolation (FIG. 5A). A total of 3 samples were sequenced, yielding 8873 cells that passed the quality control and were found to be negative for Ptprc and CD3e (FIG. 5C). The radiation conditioning did not result in the complete loss of a cell type nor the appearance of a new subset (FIG. 5C, 5D and FIG. 6E). Multiple populations showed large decreases in relative abundance, such as TEC B and endothelial cells, but these failed to reach statistical significance (FIG. 6E). The MSC compartment did however display major shifts (FIG. 5C). The stress responsive Penk+ Cdh11+ MSC were found to be significantly expanded whereas there was a dramatic reduction the frequency of the T cell supportive Postn+ MSCs (FIG. 5D). Suggesting that inefficient T cell production following cytotoxic conditioning and bone marrow transplantation, may in part be due to this observed imbalance in thymus MSC subsets.

To further probe the functional features of the MSCs post-transplantation, another GO term analysis of significantly differentially expressed genes was performed. Notably, Penk+ Cdh11+ MSC were still characterized by terms involving adipogenesis and response to various stressors, but there was also a significant enrichment for pathways inhibiting leukocyte proliferation (FIG. 5E).

Accordingly, T cell production may be further inhibited by the expansion of these cells after radiation conditioning. Postn+ MSCs on the other hand were still found to be supportive of T cells and endothelial cells (FIG. 5E and FIG. 6F) but they also displayed an augmentation of adipogenic activity. In the bone marrow it is well established that MSCs respond to irradiation by differentiating into adipocytes. Whether bone marrow adipocytes are enhancing or impeding hematopoiesis, remains contested. Thymic adipocytes, however, are not able to support T cell development, suggesting additional negative ramifications of the observed alterations of MSCs after irradiation and bone marrow transplantation.

### Example 4. Transfer of CD248− Thymic MSCs Accelerates T-Cell Production Following Radiation Conditioning

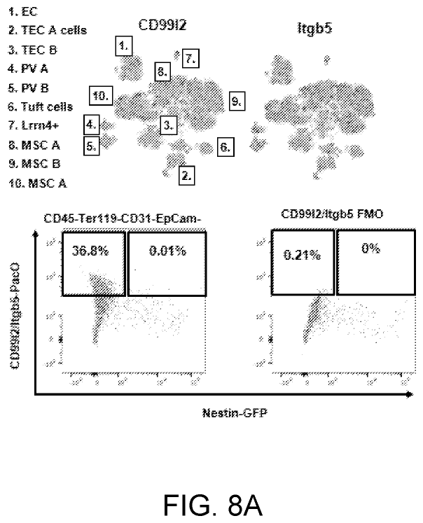

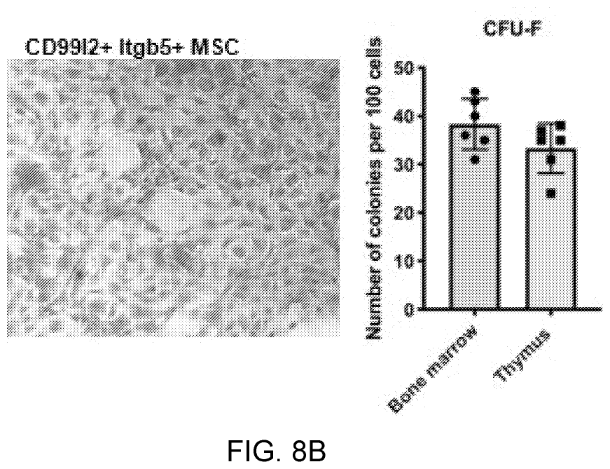

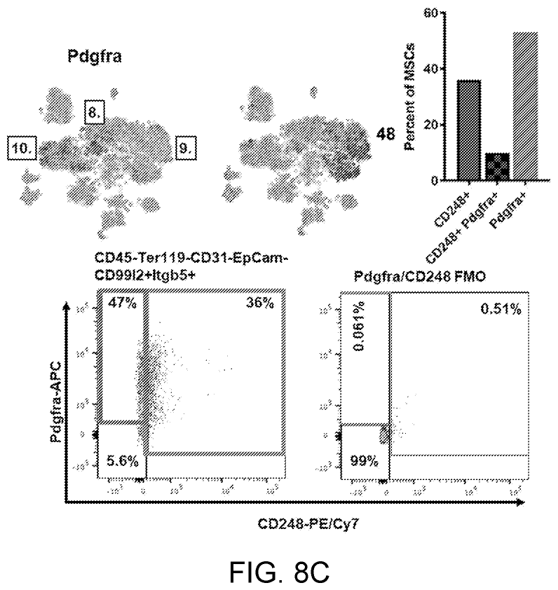

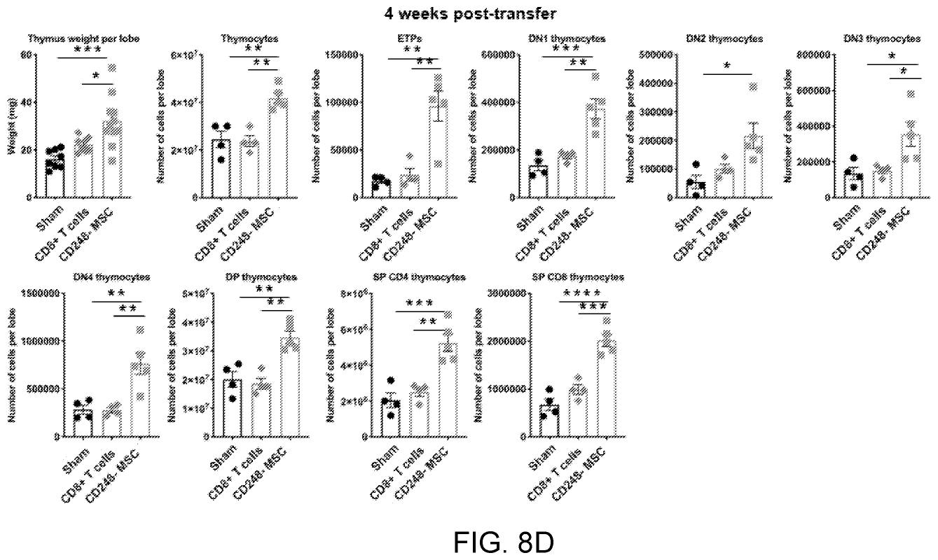

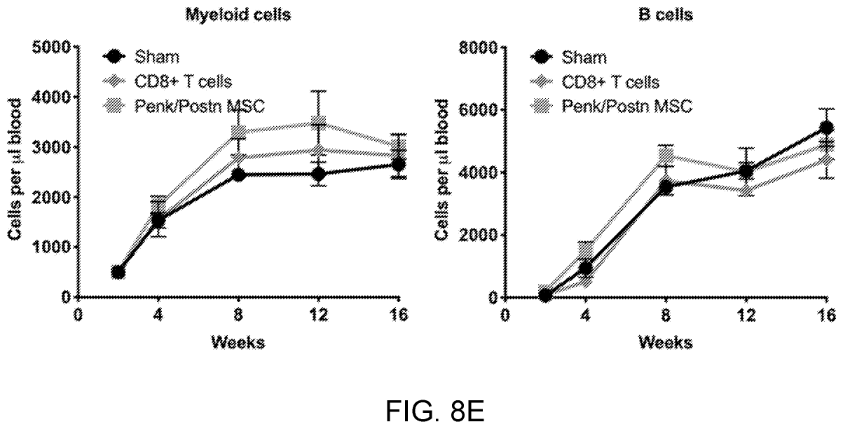

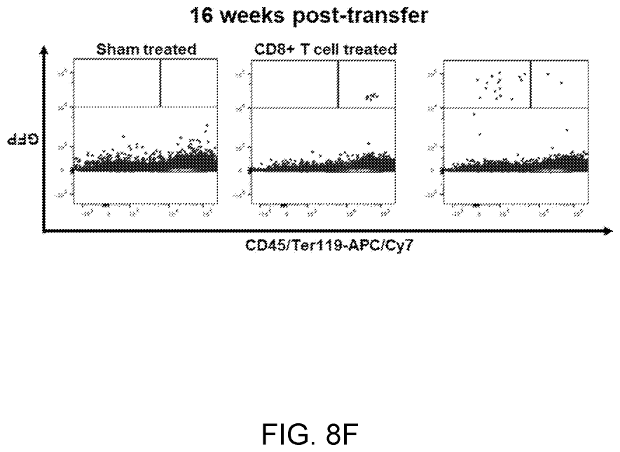

In order to test the functional significance of thymic MSCs, the scRNA sequencing data was queried for potential cell surface markers that could be used to facilitate flow cytometric sorting of the individual MSC subsets. Unfortunately, there were no suitable markers that enabled distinction between Penk+ Cdh11+ MSCs and the Postn+ population. Two markers were identified that appeared to label all MSCs while showing little overlap with perivascular cells, CD9912 and Itgb5 (FIG. 8A). The specificity for these markers within the MSC compartment was further confirmed by flow cytometric analysis, as well as sorting and plating of CD9912+ Itgb5+ thymic cells (FIGS. 8A and 8B). These cells were found to adhere to plastic and to equivalent to bone marrow MSCs in colony forming ability (FIG. 8B). Additionally, Penk+ Cdh11+ MSCs, as well as Postn+ MSCs, were found to express Pdgfra and as previously described, these cells were negative for Cd248 (FIG. 8C). Consequently, sorting CD45-Ter119-CD31-CD326-CD248-CD9912+Itgb5+Pdgfra+ cells enriched for the most T cell supportive MSCs (CD248− MSCS) while excluding the CD248+ MSCs that appeared to be of less importance.

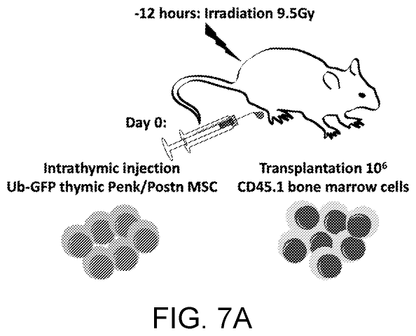

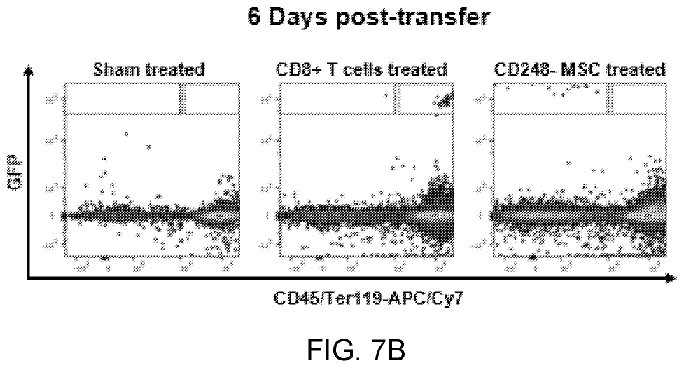

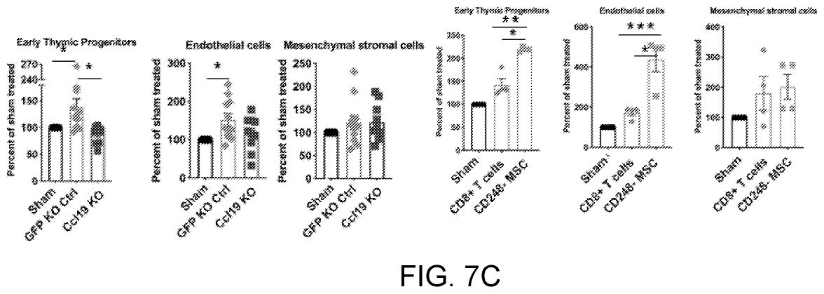

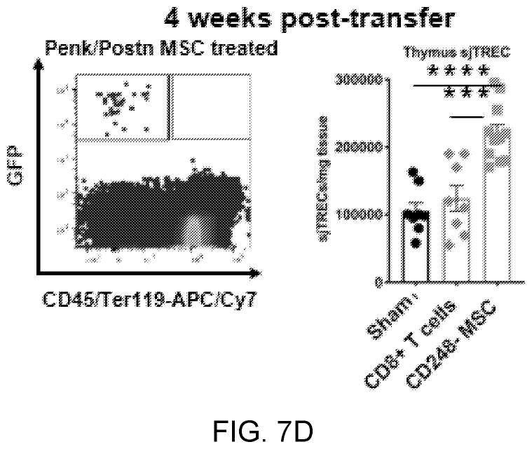

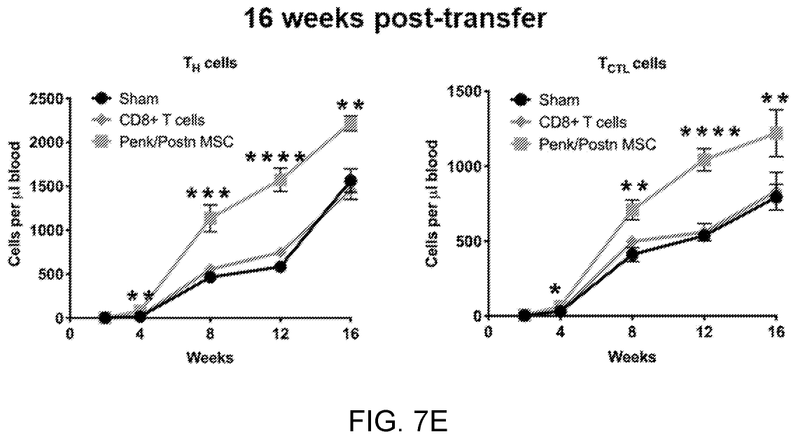

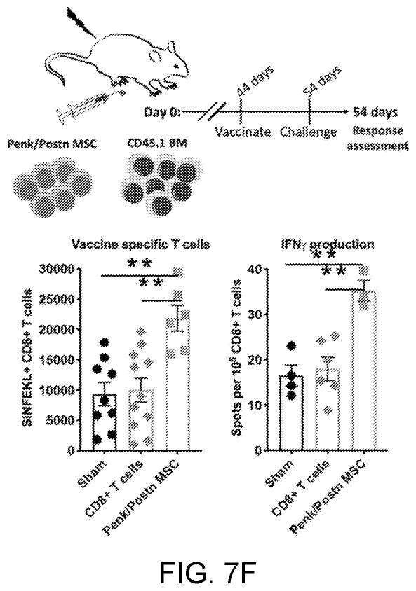

Using Ubiquitin-GFP mice as donors, CD248− MSCs were isolated and injected intrathymically in to irradiated recipients that also received a bone marrow graft (FIG. 7A). Alongside the MSC treated mice, Sham treated recipients were injected with the bone marrow, but received an intrathymic injection of PBS (FIG. 7B). In order to control for the introduction of cells into the tissue a cohort of mice was included that were given an intrathymic injection of single-positive CD8 thymocytes, a population of cells previously not implicated in thymus regeneration (FIG. 7B). Six days post-transplantation, flow cytometric analysis demonstrated that the GFP labeled CD248− MSCs persisted in the tissue (FIG. 7B). The presence of the transferred MSCs was further associated with improved numbers of both ETPs as well as endothelial cells (FIG. 7B), whereas MSC and epithelial cell numbers (data not shown) remained unchanged compared to Sham and CD8+ T cell treated mice. This indicates that that an infusion of fresh thymic CD248− MSCs after radiation conditioning can improve thymus regeneration.

One of the factors significantly enriched in thymic MSCs, Cc119, has previously been implicated in recruitment of ETPs. To determine if Cc119 expression in MSCs was necessary for the observed improvement in ETP seeding after transplantation, CD248− MSCs were isolated from Cas9-GFP expressing mice. These cells were subsequently infected with lentiviral vectors expressing guide RNAs directed towards Cc119 or the control locus GFP. Transplantation of these modified MSCs demonstrated that knockout of Cc119 abrogated the improvement in ETP recruitment following CD248 MSC treatment (FIG. 7C).

In order to determine if the increased influx of progenitors at day 6 resulted in increased de novo generation of T cells, the transplantation experiment was repeated. This time thymi were analyzed after 4 weeks. The GFP+CD248− MSCs were still found to be present in the tissue (FIG. 7C) and thymus weight as well as cellularity were significantly higher in MSC treated mice (FIG. 8D). sjTREC analysis further demonstrated that production of newly rearranged T cells was significantly improved in the mice injected with CD248− MSCs (FIG. 7C). This was further corroborated by higher numbers of cells in all stages of T cell development (FIG. 8D). Additionally, 16 weeks follow-up of transplanted mice showed that numbers of CD4+ TH cells and CD8+ Tcm cells were dramatically improved in CD248− MSC recipients (FIG. 7D), with no impact on B cells or myeloid populations (FIG. 8E). Remarkably, analysis of the thymus stromal compartment 16 weeks after the transplantation revealed that GFP+ MSCs are still surviving in the tissue (FIG. 8F).

The definitive goal of improving T cell numbers following a bone marrow transplantation is to enhance functional immunity. Transplantation recipients were therefore vaccinated against ovalbumin after 44 days (FIG. 7F). Following a re-challenge, CD248− MSC treated mice were found to have significantly improved immune responses as evidenced by increased numbers of ovalbumin specific CD8+ T_{CTL} cells, producing IFNγ (FIG. 7F). Thus, the improvements in early thymic regeneration seen after CD248− MSC transfer ultimately translate into a robust production of functional T cells.

### Example 5. Periostin+ MSCs Specifically Enhance T Cell Progenitor Recruitment

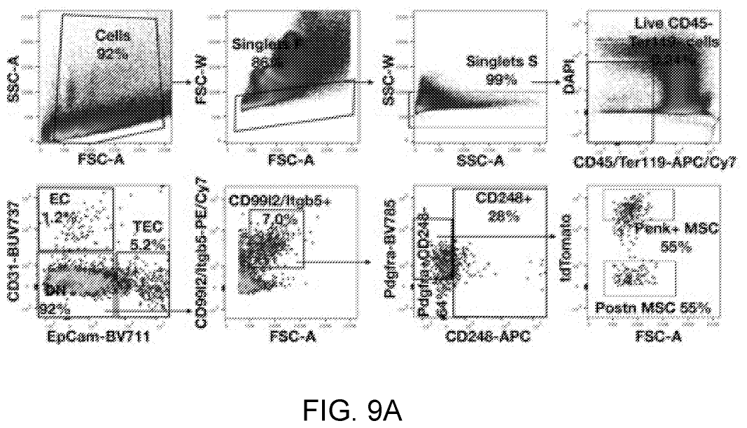

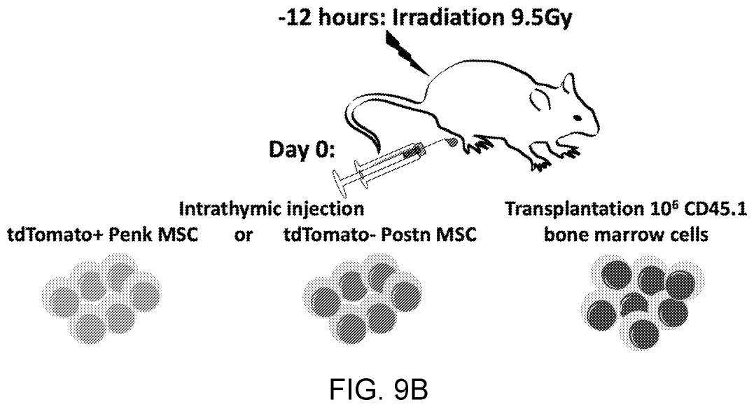

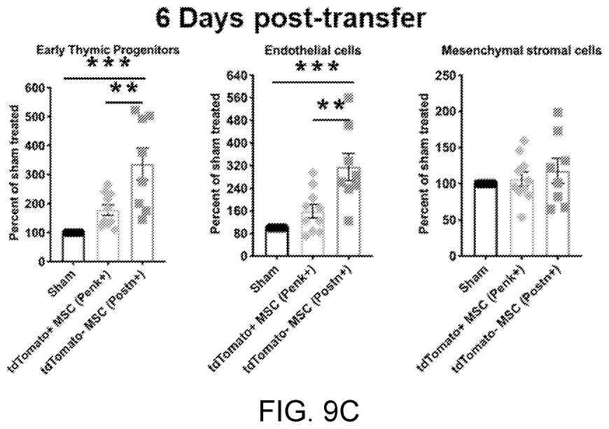

Penk-Cre mice were crossed with the Rosa26-LSL-tdTomato reporter to generate mice where Penk+ Cdh11+ and Postn MSCs could be separated. Initial flow cytometric analysis of these mice showed that the CD45-Ter119-CD31-CD326-CD248-CD9912+Itgb5+Pdgfra+ subset segregated into distinct tdTomato+(Penk+) and tdTomato− (Postn+) populations (FIG. 9A), suggesting that this reporter was faithful to the scRNA sequencing data. Indeed, transfer of tdTomato+ or tdTomato− cells in the context of bone marrow transplantation, demonstrated recipients of the presumptive Postn+ MSCs had improved ETP and endothelial cell numbers after 6 days (FIG. 9B). The effects mediated by thymic MSCs therefore appear to be contained within the Postn+ MSC population.

### REFERENCES

All patents, patent applications and publications mentioned in this specification are herein incorporated by reference to the same extent as if each independent patent and publication was specifically and individually indicated to be incorporated by reference.

1. A method for increasing the production of T cells within a T-cell producing tissue or fluid of a subject in need thereof, said method comprising administering a composition comprising mesenchymal stromal cells into a T-cell producing tissue or fluid of the subject, wherein the mesenchymal stromal cells express Periostin and Pdgfra, thereby increasing the production of T cells within the T-cell producing tissue or fluid of the subject.

2. The method of claim 1, wherein the mesenchymal stromal cells do not express Cdh11 and CD248.

3. The method of claim 1, wherein the T-cell producing tissue is thymus.

4. The method of claim 1, wherein the T-cell producing tissue is a lymphopoietic tissue.

5. The method of claim 1, wherein the T-cell producing fluid is blood.

6. The method of claim 1, wherein the subject has undergone hematopoietic stem cell transplantation.

7. The method of claim 1, wherein the subject has one or more of a condition associated with T lymphopenia, a T cell production disorder, a T cell function disorder, a distorted repertoire of T cell receptor bearing cells, an infection or a tumor.

8. The method of claim 1, wherein the mesenchymal stromal cells express Flt3 ligand (fms related receptor tyrosine kinase 3 ligand), Ccl19 (C-C motif chemokine ligand 19), BMP2 (bone morphogenetic protein 2), BMP4 (bone morphogenetic protein 4), IL-15 (interleukin 15), IL-12a (interleukin-12a), Cxcl14 (C-X-C motif chemokine ligand 14), Ccl11 (C-C motif chemokine ligand 11), (Cxcl10 C-X-C motif chemokine ligand 10), or IL-34 (interleukin 34) and combinations thereof.

9. The method of claim 1, wherein the mesenchymal stromal cells express Ccl19, Flt3 ligand and IL-15, and do not express Cdh11 and CD248.

10. The method of claim 1, wherein the mesenchymal stromal cells are autologous to the subject.

11. The method of claim 1, wherein the mesenchymal stromal cells are derived from mesenchymal stem cells or progenitors thereof.

12. The method of claim 1, wherein the mesenchymal stromal cells are derived from embryonic stem cells or progenitors thereof.

13. The method of claim 1, wherein the mesenchymal stromal cells are derived from iPS cells or progenitors thereof.

14. A method for increasing the production of T cells within a T-cell producing tissue or fluid of a subject in need thereof, said method comprising administering a composition comprising Ccl19 (C-C motif chemokine ligand 19) into a T-cell producing tissue or fluid of the subject, thereby increasing the production of T cells within the T-cell producing tissue or fluid of the subject.

15. The method of claim 14, wherein the T-cell producing tissue is thymus.

16. The method of claim 14, wherein the T-cell producing tissue is a lymphopoietic tissue.

17. The method of claim 14, wherein the T-cell producing fluid is blood.

18. The method of claim 14, wherein the subject has undergone hematopoietic stem cell transplantation.

19. The method of claim 14, wherein the subject has one or more of a condition associated with T lymphopenia, a T cell production disorder, a T cell function disorder, a distorted repertoire of T cell receptor bearing cells, an infection or a tumor.

20. A composition comprising isolated mesenchymal stromal cells expressing Periostin and Pdgfra.

21. The composition of claim 20, wherein the mesenchymal stromal cells do not express Cdh11 and CD248.

22. The composition of claim 20, wherein the mesenchymal stromal cells express Flt3 ligand (fms related receptor tyrosine kinase 3 ligand), Ccl19 (C-C motif chemokine ligand 19), BMP2 (bone morphogenetic protein 2), BMP4 (bone morphogenetic protein 4), IL-15 (interleukin 15), IL-12a (interleukin-12a), Cxcl14 (C-X-C motif chemokine ligand 14), Ccl11 (C-C motif chemokine ligand 11), (Cxcl10 C-X-C motif chemokine ligand 10), or LL-34 (interleukin 34,) and combinations thereof.

23. The composition of claim 20, wherein the mesenchymal stromal cells express Ccl19, Flt3 ligand and IL-15, and do not express Cdh11 and CD248.

24. The composition of claim 20, wherein the mesenchymal stromal cells are derived from mesenchymal stem cells or progenitors thereof.

25. The composition 20, wherein the mesenchymal stromal cells are derived from embryonic stem cells or progenitors thereof.

26. The composition of claim 20, wherein the mesenchymal stromal cells are derived from iPS cells or progenitors thereof.

27. A population of isolated stem cells capable of differentiating into mesenchymal stromal cells, wherein said mesenchymal stromal cells express Periostin and Pdgfra.

28. The population of isolated stem cells of claim 26, wherein the mesenchymal stromal cells do not express Cdh11 and CD248.

29. A composition for increasing the production of T cells within a T-cell producing tissue or fluid of a subject, said composition comprising Ccl19 (C-C motif chemokine ligand 19).
